(19) United States
(12) Patent Application Publication
(10) Pub. No.: US 2006/0068390 A1
(43) Pub. Date: Mar. 30, 2006

(54) Dna amplification and sequencing in collapsible emulsions

(76) Inventors: Daniel Tillett, New South Wales (AU); Torsten Thomas, New South Wales (AU)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/517,698

(22) PCT Filed: Jun. 13, 2003
(86) PCT No.: PCT/AU03/00746

§ 371 Date: Dec. 13, 2004

(30) Foreign Application Priority Data
Jun. 13, 2002 (AU) PS2981

(51) Int. Cl. C12Q 1/68; C12P 21/06; C12P 19/34

(52) U.S. Cl. 435/6; 435/91.2; 435/68.1

(57) ABSTRACT

The present invention relates to a method of performing a chemical reaction, in particular a small-scale chemical reaction. The method involves the use of two (or more) phases which, when formed into an emulsion, have the characteristic of being subject to “collapse” under certain physical or chemical conditions such that the discontinuous phase dispersed in the emulsion becomes a substantially continuous phase—the chemical reaction taking place in the newly-formed continuous phase.

### TECHNICAL FIELD OF THE INVENTION

The present invention relates to a method of performing a chemical reaction, in particular a small-scale chemical reaction. The method involves the use of two (or more) phases which, when formed into an emulsion, have the characteristic of being subject to “collapse” under certain physical or chemical conditions such that the discontinuous phase dispersed in the emulsion becomes a substantially continuous phase—the chemical reaction taking place in the newly-formed continuous phase.

The method is particularly applicable in the field of molecular biology since it allows submicrolitre-scale chemical and enzymatic reactions to be carried out using microlitre-scale liquid handling equipment.

### BACKGROUND

Any discussion of the prior art throughout the specification should in no way be considered as an admission that such prior art is widely known or forms part of common general knowledge in the field.

There has been a general trend to performing chemical and enzymatic reactions on a smaller and smaller scale. This has been driven by both the high cost of many reagents and the increased sensitivity of modern analytical equipment. For example, the cost of DNA sequencing reagents has been reported to be responsible for greater than 30% of the total cost of obtaining sequence data from a specific DNA template (Nakane et al., 2001). Consequently, many laboratory reactions are now performed on a microlitre-scale (e.g. 3-10 microlitres). While this has led to significant cost savings, there are two major technical hurdles limiting further reductions in reaction scale. The first of these is evaporation, in particular for those reactions that involve sample heating (e.g. PCR and cycle DNA sequencing). The rate of evaporation is a function at least of four factors: the temperature of the sample, the temperature of the environment, the humidity of the environment, and the surface to volume ratio of the sample. While the first three of these factors can be controlled to some degree by careful experimental design, an increase in the surface to volume ratio is an inherent consequence of smaller volumes. Therefore, as the volume of a sample or reagent solution becomes smaller, it becomes increasingly difficult to prevent evaporation of the sample.

Mineral oil overlays have been used in polymerase chain reaction (PCR) protocols in order to avoid evaporation of the sample. The reaction is prepared in the standard manner and an aliquot of mineral oil is added to the final reaction mix prior to temperature cycling (Saiki et al., 1988). Since the aqueous phase remains beneath the mineral oil overlay during the PCR, this can be an effective method of preventing evaporation—particularly during the DNA denaturation step that takes place at high temperatures. It does not, however, address or alleviate the problem of dispensing reagents in small volumes for small-scale chemical reactions.

The second major hurdle faced in reducing the scale of chemical and enzymatic reactions is the accuracy of the fluid handling equipment found in most laboratories. The transfer of fluids from one container to another is one of the most common tasks performed in a typical chemical or biological laboratory. For example, the process of DNA sequencing requires the addition of separate solutions of template, primer, buffer, enzyme and nucleotides, to the reaction vessel. These processes are currently performed either by hand using manual pipettors, or automatically using robotic pipetting instruments. However, standard manual pipettors cannot accurately transfer volumes of less than one microlitre (Meldrum et al., 2000). Further, automated robotic liquid handling systems are often less accurate than manual systems, thus effectively limiting high throughput applications to reaction volumes of greater than five microlitres (Meldrum et al., 2000).

To overcome these limitations high precision systems have been developed. Seubert et al. describe in U.S. Pat. Nos. 5,785,926 and 6,218,193 a high precision, small volume fluid processing system employing open-ended capillaries to meter, aliquot and mix nanolitre-scale volumes of sample fluids and reagents. A similar system, based on disposable pipet tips, has been described by Nakane et al. for performing DNA sequencing reaction in volumes of less than one microlitre (Nakane et al., 2001). Wiktor describes in U.S. Pat. No. 6,323,129 a piezoelectric pipetting system capable of accurate liquid transfers as small as 100 nanolitres. Hadd et al. describes a system for performing 500 nanolitre DNA sequencing reaction in fused-silica capillaries (Hadd et al., 2000).

While these systems are capable of performing nanolitre-scale reactions, they require the use of highly specialised and expensive equipment not generally available to most investigators. Furthermore, these systems require the use of high precision consumables, such as glass capillaries and pipet tips, which are often significantly more expensive than standard laboratory consumables. Finally, these systems present difficulties in workflow integration as the reactions are performed in non-standard sized reaction vessels.

Therefore, a need exists for a system capable of performing chemical and enzymatic reactions on a nanolitre-scale that employs standard microlitre-scale fluid handling equipment and reaction vessels. The system should also preferably prevent evaporation of the reaction, both during set up and during the reaction, especially those applications requiring high temperature incubation (i.e. DNA sequencing and PCR). Preferably, the system should also be capable of being integrated into current high-throughput applications (e.g. DNA sequencing and PCR).

It is an object of the present invention to provide a method that will ameliorate at least some of the deficiencies of the prior art or will provide a useful alternative.

### SUMMARY OF THE INVENTION

It has surprisingly been found that a chemical reaction can be performed by introducing a discontinuous first phase comprising at least one of the reactants, into a continuous second phase by forming an emulsion, subjecting the emulsion to a physical or chemical change such that the discontinuous first phase coalesces to a substantially continuous phase and providing conditions in which the chemical reaction can take place in the newly formed continuous first phase.

One of the major advantages of such a method is that it is especially suited to accommodate the use of microlitre-scale equipment to perform submicrolitre-scale reactions—in particular in applications relevant to molecular biology. In fact, the present invention provides a system that can be used to perform chemical and enzymatic reactions on a nanolitre-scale utilising currently available microlitre-scale fluid handling equipment and reaction vessels.

Specifically, the present invention allows the transfer of the reaction components to the reaction vessel in the form of one or more emulsions in which, for example, a relatively large inert phase may form the continuous phase and a relatively small aqueous phase may form the discontinuous phase. Once the emulsions have been transferred to the reaction vessel in the desired quantities, a physical or chemical change is induced, for example by a change in the physical conditions or by the addition of other chemical compounds, such that the aqueous and inert phases separate and a substantially continuous aqueous phase is formed. Conditions are provided such that the desired chemical reaction occurs in the aqueous phase. The use of emulsions of different discontinuous phase:continuous phase ratios allows the reaction to be scaled to the sensitivity of the analytical equipment used rather than the fluid handling equipment available. For example, a 500-nanolitre reaction can performed using five microlitres of a 10:1 inert:aqueous phase emulsion.

This invention may further avoid introducing potentially detrimental effects associated with reagent dilution that may otherwise cause the chemical reaction to fail (e.g. in DNA sequencing). Creation of the emulsion does not dilute the concentration of reagents contained within the discontinuous phase. Thus this makes the present invention highly suitable for applications in which the concentration of the reactants is critical (e.g. most enzymatic reactions).

The present invention further provides a system capable of preventing significant sample evaporation, especially for those applications that require high temperature incubations, such as polymerase chain reactions (PCRs).

In addition, the present invention further provides a system that can be integrated into current high-throughput systems.

Other advantages of the present invention will become apparent from the description that follows.

Accordingly, in a first aspect, the present invention provides a method of performing a chemical reaction between reactants comprising:

(a) subjecting an emulsion comprising
(i) a discontinuous first phase in which at least one of the reactants is present; and
(ii) a substantially continuous second phase, to a physical or chemical change such that a substantially continuous phase is formed from the discontinuous phase; and

(b) providing conditions in which the chemical reaction between the reactants takes place.

Preferably, the discontinuous first phase is an aqueous phase and preferably, the continuous second phase is an inert or an organic phase. However, it will be clear to the skilled addressee that the first phase maybe an inert or organic phase and the second phase may be an aqueous phase.

According to a second aspect, the present invention provides a method of performing a chemical reaction between reactants in an aqueous phase comprising:

(a) subjecting an emulsion comprising
(i) a discontinuous aqueous phase in which at least one of the reactants is present; and
(ii) a continuous inert phase, to a physical or chemical change such that a substantially continuous aqueous phase is formed; and

(b) providing conditions in which the chemical reaction between the reactants takes place.

Preferably, the chemical reaction is a reaction selected from the group consisting of: DNA sequencing, Polymerase Chain Reaction (PCR), Rolling Circle Amplification (RCA), Ligase Chain Reaction (LCR), Rapid Amplification of cDNA Ends (RACE), reverse-transcriptase PCR (RT-PCR), DNA fingertyping, DNA genotyping, endonuclease-restriction digest, DNA ligation, DNA phosphorylation, DNA methylation, DNA labelling, ribonucleic acid (RNA) digestion, proteolytic digestion, and protein modification. Protein modification may be by, for example, glycosylation or phosphorylation. More preferably, the chemical reaction is DNA sequencing or PCR. It is clear, however, that the method of the invention is not limited to the reactions mentioned above and can be used for any suitable chemical reaction. The skilled addressee will be able to determine, based on the nature of the reactants, the types of reactions for which the method of the invention would be suitable.

Preferably, the reactants are selected from the group consisting of: DNA, RNA, mRNA, proteins, enzymes, salts, radioactive isotopes, carbohydrates, or other organic and inorganic molecules although it will be clear that other reactants are also possible and will be easily identifiable by the skilled addressee. When the reactant is DNA, it may be, for example, gDNA, cDNA, mDNA, primer DNA, plasmid DNA or a PCR product. When the reactant is an enzyme, it may be a DNA polymerase, RNA polymerase, reverse transcriptase, restriction endonuclease, DNA methylase, polynucleotide kinase, nucleotide transferase, DNA ligase, RNA ligase, protease, or other DNA, RNA or protein modifying enzyme.

Clearly, as described in the examples below (eg. Examples 5, 6 and 10), one or other of the reactants may not necessarily be present in the continuous OR discontinuous phase when the continuous and discontinuous phases are combined. For example, when a polymerase chain reaction (PCR) or DNA sequencing reaction is set up, the template DNA may initially be present in a dry form in the vessel into which the continuous and discontinuous phases are placed. At least a portion of the template DNA will, over time, migrate to the aqueous phase—the extent and rapidity of this migration will depend on the reaction conditions and it may occur before, during and/or after the collapse of the emulsion.

Preferably, the aqueous phase is in a submicrolitre volume and more preferably, it is in a nanolitre volume ranging from 5 to 1000 nanolitres. The final composition of the aqueous phase can be chosen to allow for optimal conditions for a given chemical or biochemical reaction to occur.

The person skilled in the art will recognise that the invention is not limited to any particular type of emulsion provided it allows for the formation of a substantially continuous phase to be formed from the discontinuous phase when subjected to the physical or chemical change. A mixture of emulsions may also be used.

Preferably, the emulsion is prepared by combining a first and second emulsion wherein
(a) the first emulsion comprises a first aqueous phase and a first inert phase wherein the first aqueous phase comprises a first reactant; and
(b) the second emulsion comprises a second aqueous phase and a second inert phase wherein the second aqueous phase comprises a second reactant.

In a particular preferred embodiment, the first and second inert phases are the same but the first and second aqueous phases are different. It will be clear to the person skilled in the art that such a system can be used to effectively control the timing of the chemical reaction, i.e. given that the aqueous phases are different, the conditions can be arranged such that the reactants only combine and react once the substantially continuous phase has been produced and this can be controlled by timing the physical or chemical change.

In another embodiment, the emulsion comprises a single inert phase and two or more different aqueous phases comprising different reactants. This type of emulsion can be of use, for example, when one reactant is required in a small but accurate amount and a second reagent can be provided in a larger, more easily manipulated amount. The first reactant can be prepared in an emulsion comprising an inert phase and an aqueous phase, the aqueous phase comprising the reactant. The second reactant (in aqueous phase only) can be added to the emulsion. Upon being subjected to the physical or chemical change, the aqueous phases will be combined in the substantially continuous phase.

It will also be clear to the skilled addressee that when two or more emulsions are used, the inert phases of the emulsions may be different. If the inert phases are different, they must be compatible such that they still allow for the formation of a substantially continuous aqueous phase when subjected to the physical or chemical change.

It is well within the competence of the skilled addressee to identify suitable inert phases. It will be understood that any suitable inert phase may be used provided that when combined with a discontinuous aqueous phase, a physical or chemical change can induce the collapse of the inert phase such that a substantially continuous aqueous phase is formed.

A desirable inert phase will not adversely affect the chemical reaction to be performed in the aqueous phase. The choice of inert phase used should therefore be made by considering the reactants, and the type of chemical reaction to be performed, and choosing the inert phase that will have the lowest solubility in the aqueous phase and the least adverse effect on the reactants and chemical reaction.

Suitable inert phases may include, for example, non-polar water-immiscible compounds or compositions such as, for example, hydrocarbons. The hydrocarbons may be of various chain-lengths (e.g. pentane, hexane, heptane, octane, nonane, decane, dodecane, hexadecane, octadecane, eicosane, squalane and the like), various branching (e.g. 7-methyl-1,6-octadiene or 2,2,4-trimethylpentane), various bonding (e.g 1-dodecene, 1-hexadecane, squalene), or ring structure (e.g. cyclohexane or propylcyclohexane) and may have a variety of substituents eg. chloro fluoro hydrocarbons.

Other inert phases which may be suitable in some applications of the present invention are the various natural or non-natural mixtures of hydrocarbons of defined, incompletely defined or undefined composition, such as mineral oil or petroleum oil, that cannot be directly mixed with water.

In some applications, a polysiloxane compound may be employed as the inert phase. Volatile or non-volatile polysiloxanes may be useful, including compounds such as cyclic dimethyl polysiloxanes having from three to six silicon atoms, such as cyclomethicone, as well as suitable linear or branched polysiloxanes.

Preferably, the inert phase is selected from the group consisting of: mineral oil, hexadecane, dodecane and n-hexane.

According to the present invention, the aqueous phase and the inert phase are mixed such that preferably a quasi-homogenous emulsion is formed between them. Formation of emulsions can be facilitated and stabilised by the presence of surface active agents (surfactants). Three general types of surfactants exist—non-ionic, ionic, and zwitterionic (Helenius et al., 1979; Neugebauer, 1990).

Preferably, the emulsion comprises a surfactant.

Examples of non-ionic surfactants which may be useful in the present invention include, but are not limited to: APO-10, APO-12, BRIJ-35, C8E6, C10E6, C10E8, C12E6, C12E8 (Atlas G2127), C12E9, C12E10 (Brij 36T), C16E12, C16E21, cyclohexyl-n-ethyl-beta-D-maltoside, cyclohexyl-n-hexyl-beta-D-maltoside, cyclohexyl-n-methyl-beta-D-maltoside, n-decanoylsucrose, n-decyl-beta-D-glucopyranoside,n-decyl-beta-D-maltopyranoside, n-decyl-beta-D-thiomaltoside, n-dodecanoylsucrose, n-dodecyl-beta-D-glucopyranoside, n-dodecyl-beta-D-maltoside, genapol C-100, genapol X-80, genapol X-100, HECAMEG, heptane-1,2,3-triol, n-heptyl-beta-D-glucopyranoside, n-heptyl-beta-D-thioglucopyranoside, LUBROL PX, MEGA-8 (ocatanoyl-N-methylglucamide), MEGA-9 (nonanoyl-N-methylglucamide), MEGA-10 (decanoyl-N-methylglucamide), n-nonyl-beta-D-glucopyranoside, Nonidet P-10 (NP-10), Nonidet P-40 (NP-40), n-octanoyl-beta-D-glucoslyamine (NOGA), n-octanoylsucrose, n-octyl-alpha-D-glucopyranoside, n-octyl-beta-D-glucopyranoside, n-octyl-beta-D-maltopyranoside, PLURONIC F-68, PLURONIC F-127, THESIT, TRITON X-100 (tert-C8-Ø-E9.6;like NP-40), TRITON X-100 hydrogenated, TRITON X-114 (tert-C8-Ø-E7-8), TWEEN 20 (C12-sorbitan-E20;Polysorbate 20), TWEEN 40 (C16-sorbitan-E20), TWEEN 60 (C18-sorbitan-E20), TWEEN 80 (C18:1-sorbitan-E20), n-undecyl-beta-D-maltoside, cetearyl alcohol, hydrogenated tallow alcohol, lanolin alcohols, palmamide, peanutamide MIPA, PEG-50 tallow amide, cocamidopropylamine oxide, lauramine oxide, PEG-8 dilaurate, PEG-8 laurate, PEG-4 caster oil, PEG-120 glyceryl stearate, triolein PEG-6 esters, glycol stearate, propylene glycol ricinoleate, glyceryl myristate, glyceryl palmitate lactate, polyglyceryl-6 distearate, polyglyceryl-4 oleyl ether, methyl gluceth-20 sesquistearate, sucrose distearate, polysorbate-60, sorbitan sequiisostearate, trideceth-3 phosphate, trioleth-8 phosphate, ceteareth-10, nonoxynol-9, PEG-20 lanolin, PPG-12-PEG-65 lanolin oil, dimethicone copolyol, meroxapol 314, poloxamer 122, PPG-5-ceteth-20 and lauryl glucose.

Examples of ionic surfactants which maybe useful in the present invention include, but are not limited to: caprylic acid (n-octanoate), cetylpyridinium chloride, CTAB (Cetyltri-methylammonium bromide), cholic acid, decanesulfonic acid, deoxycholic acid, dodecyltrimethyl-ammonium bromide, glycocholic acid, glycodeoxycholic acid, lauroylsarcosine (sarkosyl), lithium n-dodecyl sulfate, lysophosphatidyl-choline, sodium n-dodecyl sulfate (SDS, lauryl sulfate), taurochenodeoxy-cholic acid, taurocholic acid, taurodehydrocholic acid, taurodeoxycholic acid, taurolithocholic acid, tauroursodeoxycholic acid, tetradecyltrimethyl-ammonium bromide (TDTAB), TOPPS, di-TEA-palmitoyl aspartate, sodium hydrogenated tallow glutamate, palmitoyl hydrolysed milk protein, sodium cocoyl hydrolysed soy protein, TEA-abietoyl hydrolysed collagen, TEA-cocoyl hydrolysed collagen, myristoyl sarcosine, TEA-lauroyl sarcosinate, sodium lauroyl taurate, sodium methyl cocoyl taurate, lauric acid, aluminium stearate, cottonseed acid, zinc undecylenate, calcium stearoyl lactylate, laureth-6 citrate, nonoxynol-8 carboxylic acid, sodium trideceth-13 carboxylate, DEA-oleth-10 phosphate, dilaureth-4 phosphate, lecithin, sodium cocoyl isethionate, sodium dodecylbenzene sulfonate, sodium cocomonoglyceride sulfonate, sodium C12-14 olefin sulfonate, sodium C12-15 pareth-15 sulfonate, sodium lauryl sulfoacetate, dioctyl sodium sulfosuccinate, disodium oleamido MEA-sulfosuccinate, ammonium laureth sulfate, sodium C12-13 pareth sulfate, MEA-lauryl sulfate, cocamidopropyl dimethylamine lactate, dimethyl lauramine, soyamine, stearyl hydroxyethyl imidazoline, PEG-cocopolyamine, PEG-15 tallow amine, benzalkonium chloride, quatemium-63, oleyl betaine, sodium lauramidopropyl hydroxyphostaine, cetylpyridinium chloride, isostearyl ethylimidonium ethosulfate, cocamidopropyl ethyldimonium ethosulfate, hydroxyethyl cetyldirnonium chloride, quaternium-18 and cocodimonium hydroxypropyl hydrolysed hair keratin.

Examples of zwitterionic surfactants which may be useful in the present invention include, but are not limited to: BigCHAP, CHAPS, CHAPSO, DDMAU, EMPIGEN BB (N-dodecyl-N,N-dimethylglycine), lauryldimethylamine oxide (LADAO, LDAO, Empigen OB), ZWITTERGENT 3-08, ZWITTERGENT 3-10, ZWITTERGENT 3-12 (3-dodecyl-dimethylammonio-propane-1-sulfonate), ZWITTERGENT 3-14, ZWITTERGENT 3-16, disodium cocoamphocarboxymethylhydroxy-propylsulfate, disodium cocoamphodipropionate, sodium cocoamphoacetate, sodium lauroampho PG-acetate phosphate, sodium tallow amphopropionate, sodium undecylenoamphopropionate, aminopropyl laurylglutamide, dihydroxyethyl soya glycinate and lauraminopropionic acid.

Other examples of suitable surfactants may be found in standard reference texts such as the Cosmetics and Toiletries Surfactant Encyclopedia ((1996) M. M. Rieger, 2^{nd} Ed., published by Allured Publishing Corporation, Carol Stream, Ill. 60188 2787, USA or McCutcheon's Emulsifiers and Detergents (2002) published by MC Publishing Co., Glen Rock, N.J. 07452, USA).

Preferably, the surfactant is TRITON X-100 or TRITON-X114.

Preferably, the physical or chemical change is a change in temperature, pressure or exposure to a chemical compound. It will be clear to the skilled addressee that any physical or chemical change that results in the formation of a substantially continuous aqueous phase from an emulsion of an inert phase and a discontinuous aqueous phase is also contemplated. Preferably, the physical change is a change in temperature. In one embodiment, the chemical change is the addition of glycerol.

When the chemical reaction is a DNA sequencing or PCR reaction, the inert phase preferably comprises mineral oil and the surfactant preferably comprises TRITON X-100 or TRITON-X114.

The appropriate ratio of the discontinuous phase:continuous phase (eg. aqueous phase:inert phase) will be easily determined by the skilled addressee upon reasonable trial and error. For example, in some applications, ratios of aqueous to inert phases in the range of 1:4 to 1:19 will be useful. When the chemical reaction is a DNA sequencing reaction, it is preferably performed using an aqueous phase between two microlitres and 500 nanolitres and a final volume of emulsion of 10 microlitres.

It will be understood that although step (b) of the method is mentioned last, the environmental conditions in which the chemical reaction between the reactants takes place may be present at any time during the performance of the method eg. the conditions may be present when the emulsion is prepared and/or when the substantially continuous aqueous phase is formed and/or at some time thereafter. For example, if the chemical reaction takes place at a particular temperature, eg. 37° C., two emulsions each comprising a reactant could be mixed at this temperature, the substantially continuous aqueous phase could be formed at this temperature by the addition of a chemical and the chemical reaction could be allowed to proceed at the same temperature.

It will be clear to the skilled addressee that the substantially continuous aqueous phase may be removed from the inert phase or vice versa prior to being subjected to conditions at which the chemical reaction proceeds or, alternatively, that the aqueous phase and the inert phase may be submitted to the reaction conditions together. Removal of the inert phase may be by, for example, manually removing it by suction (eg. using a pipette). Alternatively, removal of the inert phase may be by changing the environmental conditions such that the inert phase evaporates or vaporises leaving the aqueous phase. Inert phases that may be suitable in these applications include n-hexane or chloro fluoro hydrocarbons.

According to a third aspect, the present invention provides a method of performing a chemical reaction between at least two reactants in an aqueous solution comprising:

(a) combining a first emulsion in which an aqueous solution comprising a first reactant is emulsified in a first inert phase, with a second emulsion in which an aqueous solution comprising a second reactant is emulsified in a second inert phase;

(b) subjecting the mixture to a physical or chemical change such that the emulsions collapse and the emulsified aqueous solution coalesces into a substantially single or substantially continuous aqueous phase;

(c) subjecting the aqueous phase to conditions in which the chemical reaction between the reactants takes place.

According to a fourth aspect, the present invention provides a method of performing a chemical reaction between reactants in an organic phase comprising:

(a) subjecting an emulsion comprising
(i) a discontinuous organic phase in which the reactants are present; and
(ii) a continuous aqueous phase, to a physical or chemical change such that a substantially continuous organic phase is formed; and

(b) providing conditions in which the chemical reaction between the reactants takes place.

According to a fifth aspect, the present invention provides a method of performing a chemical reaction between at least two reactants in an organic solution comprising:

(a) combining a first emulsion in which an organic solution comprising a first reactant is emulsified in a first aqueous phase, with a second emulsion in which an organic solution comprising a second reactant is emulsified in a second aqueous phase;

(b) subjecting the mixture to a physical or chemical change such that the emulsions collapse and the emulsified organic solution coalesces into a substantially single or substantially continuous organic phase;

(c) subjecting the organic phase to conditions in which the chemical reaction between the reactants takes place.

The skilled addressee will understand that although the above methods are designed to alleviate problems associated with small-scale chemical reactions, and in particular reactions performed at the nanolitre level, the invention is not confined to use in sub-microlitre reactions and can be used in any application in which it is required or desired (for any reason) to have a relatively high volume of a reactant at the time it is included in a reaction mixture and a relatively low volume of the reactant at the time it is reacted.

In the context of the present invention, the term “emulsion” includes any solution in which a discontinuous first phase is dispersed in a continuous second phase. An emulsion in the context of the present application can be produced by any means, including physical agitation of the phases, the addition of surfactants to the phases, sonication or spraying. When reference is made in the specification to the emulsion “collapsing” this is the point at which the discontinuous first phase coalesces to become a substantially continuous first phase. “Collapse” may be brought about by any physical or chemical means including a shift in temperature or pressure or exposure to a chemical compound. “Collapse” may also include vaporisation or evaporation of the second phase.

In the context of the present invention, the term “inert phase” includes a compound or composition the primary function of which is to act as a “bulking” agent. For example, when the chemical reaction takes place in an aqueous phase, the inert phase preferably is substantially immiscible with water or has a low partition coefficient in water. Typically, the lower the partition coefficient in water the better but this characteristic will have to be balanced with consideration of other parameters such as the types of reactants and reaction conditions required. The skilled addressee will readily determine the degree of water solubility of the inert phase tolerable for any particular application.

In other applications, for example, in which the chemical reaction takes place in an organic phase, the inert phase could be water.

In the context of the present invention, the phrase “discontinuous phase” refers to a phase that is predominantly dispersed in another phase eg. by being emulsified.

In the context of the present invention, the phrase “substantially continuous aqueous phase” refers to a phase that is predominantly in a continuous phase.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### BRIEF DESCRIPTION OF THE DRAWINGS

The invention may be more fully understood by reference to the accompanying figures to this application.

FIG. 1 The DNA sequence of the pCR®-Blunt II-TOPO® cloning vector (SEQ ID No. 1; Invitrogen, Carlsbad, Calif., USA.) The binding-sites of the M13 outer forward (−47) and outer reverse (−48) primers are underlined.

FIG. 2 Agarose gel showing the effect of TRITON X-100 titration on the product formation during a PCR reaction. Sample 1 is derived from the control PCR reaction and samples 2-6 are derived from 0.5, 1, 2, 4, or 8% Triton-X 100 in the reaction mix, respectively. The samples labelled 1 Kb+ and λ are marker lanes consisting of the 1 Kb plus DNA ladder (Invitrogen, Carlsbad, Calif., USA) and the Lambda DNA/Hind III Marker (GeneWorks, South Australia.).

FIG. 3: Control of the interaction of ethidium bromide with DNA by inducing emulsion phase separation. The left tube contains dried DNA and an intact ethidium bromide containing emulsion. The middle tube contains dried DNA and a collapsed ethidium bromide containing emulsion. The right tube contains no DNA and a collapsed ethidium bromide containing emulsion.

FIG. 4: Agarose gel showing a PCR reaction in an emulsion reaction mix of Triton-X 100 and mineral oil. Sample 1 is the positive control PCR (no emulsion), samples 2 to 6 are derived from emulsion made from mineral oil and 0.5, 1, 2, 4, or 8% TRITON X-100, respectively. The samples labelled 1 Kb+ and λ are marker lanes consisting of the 1 Kb plus DNA ladder (Invitrogen, Carlsbad, Calif., USA) and the Lambda DNA/Hind III Marker (GeneWorks, South Australia).

FIG. 5.1: DNA sequence of the pGEM-3Zf (+) plasmid (SEQ ID No. 2) with the binding-site of the M13 (−20) forward sequencing primer underlined.

FIG. 5.2: DNA sequence of the M13mp18 (+) strand plasmid (SEQ ID No. 3) with the binding-site of the M13 (−20) forward sequencing primer underlined.

FIG. 5.3: DNA sequence of a PCR product (SEQ ID No.4) with the binding-site of the M13 (−20) forward sequencing primer underlined.

FIG. 5.4: 377 Electropherogram of a non-emulsion control DNA sequencing reaction performed using pGEM-3Zf (+) plasmid as the template DNA.

FIG. 5.5: 377 Electropherogram of a DNA sequencing reaction performed in a Triton X-100/mineral oil emulsion using pGEM-3Zf (+) plasmid as the template DNA. For further details see the text body.

FIG. 5.6: 377 Electropherogram of a DNA sequencing reaction performed in a Triton X-100/mineral oil emulsion using the M13mp18 (+) strand plasmid as the template DNA. For further details see the text body.

FIG. 5.7: 377 Electropherogram of a DNA sequencing reaction performed in a Triton X-100/mineral oil emulsion using a PCR product as the template DNA. For further details see the text body.

FIG. 6.1: DNA sequence of the pUC18 plasmid (SEQ ID No. 5) with the binding-site of the primer pGemEcoRV underlined.

FIG. 6.2: Electropherogram of a DNA sequencing reaction performed in a hexadecane/triton X-114 emulsion. For further details see the text body.

FIG. 6.3: Electropherogram of a DNA sequencing reaction performed in a mineral oil/octanol/triton X-114 emulsion. For further details see the text body.

FIG. 6.4: Electropherogram of a DNA sequencing reaction performed in a mineral oil/octanol/triton X-100 emulsion. For further details see the text body FIG. 7.1: Electropherogram of a DNA sequencing reaction performed in a diluted standard system. For further details see the text body.

FIG. 7.2: Electropherogram of a DNA sequencing reaction performed in an emulsion system. For further details see the text body.

FIG. 8: Collapse of an emulsion and removal of the inert phase from the sample. The left tube contains the intact emulsion with the aqueous phase homogenously dispersed as indicated by the red colour. The middle tube contains the collapsed emulsion after heat treatment with the aqueous phase (red) overlayed by the inert phase (clear). The right tube contains solely the aqueous phase after the inert phase has been evaporated.

FIG. 9. Kpn I digest of pGEM3Zf(+) in a chemically collapsed emulsion. Lane 1: The 90% intact non-collapsed emulsion control reaction (Sample 1). Lane 2: The thermally collapsed emulsion reaction (Sample 2). Lane 3: The chemically collapsed emulsion reaction (Sample 3). The relative mobilities of the circular and linear forms of the plasmid are indicated.

FIG. 10. Electropherogram of a DNA sequencing reaction performed in a collapsible emulsion with diluted sequencing chemistry. For further details see the text body.

FIG. 11. Electropherogram of a DNA sequencing reaction performed in a collapsible emulsion with liquid DNA template addition. For further details see the text body.

FIG. 12.1. Electropherogram of a DNA sequencing reaction performed using a 1 microlitre reaction volume and a 5 microlitre mineral oil overlay. For further details see the text body.

FIG. 12.2. Electropherogram of a DNA sequencing reaction performed using a 1 microlitre reaction volume and a 10 microlitre mineral oil overlay. For further details see the text body.

FIG. 12.3. Electropherogram of a DNA sequencing reaction performed using a 1 microlitre reaction volume and a 10 microlitre mineral oil overlay. For further details see the text body.

FIG. 12.4. Electropherogram of a DNA sequencing reaction performed using a 1 microlitre reaction volume and a 10 microlitre mineral oil overlay. For further details see the text body.

### DETAILED DESCRIPTION OF THE INVENTION

The present invention provides a method of performing chemical reactions, in particular reactions requiring reactants at volumes less than can be handled accurately using standard liquid-handling equipment. The invention allows, for example, the transfer of reaction components as an emulsion comprising a discontinuous first phase and a continuous second phase. For example, the discontinuous first phase may be an aqueous phase comprising reactants and the continuous second phase may be an inert phase. At the appropriate time, a physical or chemical change in the conditions can be induced such that the phases separate and a substantially continuous phase is formed from the discontinuous first phase, thus allowing the reaction to take place in this newly-formed continuous first phase.

Importantly, the present invention allows the use of standard fluid handling equipment to perform reactions in the nanolitre-scale. The scale of the resultant reaction is governed by the volume of the discontinuous first phase contained within the emulsion, not by the volume of the continuous second phase. Thus, by the use of emulsions of high ratios of continuous second phase:discontinuous first phase, it is possible to perform aqueous reactions on a nanolitre-scale. For example, a 500-nanolitre reaction can be performed using five microlitres of a 10:1 inert:aqueous phase emulsion.

When an aqueous phase is the discontinuous first phase, it may, for example, contain water-soluble compounds such as salts or other hydrophilic (macro-) molecules. The final composition of the aqueous phase can be chosen to allow for optimal conditions for a given chemical or biochemical reaction to occur.

In the examples provided herewith, the composition of the continuous second phase i.e. the inert phase, is chosen so as not to interfere to any detectable degree with reactants in the discontinuous first phase i.e. the aqueous reaction.

It is contemplated that the present invention may be used in a wide variety of aqueous reactions. Examples of reactions that have been proven useful in various applications in the field of molecular biology, biotechnology, medical, pharmaceutical and agricultural technology are: DNA sequencing, Polymerase Chain Reaction (PCR), Rolling Circle Amplification (RCA), Ligase Chain Reaction (LCR), Rapid Amplification of cDNA Ends (RACE), reverse-transcriptase PCR (RT-PCR), DNA fingertyping and genotyping, endonuclease-restriction digest, DNA ligation, DNA phosphorylation and methylation, DNA labelling, ribonucleic acid (RNA) digest, proteolytic digests, and protein-modification (e.g. glycosylation, phosphorylation) (Ausubel et al., 1998). Other methods for which the present invention will be useful will be recognised by the skilled addressee and fall within the scope of the present invention.

A large number of detection methods have been developed to analyse the results of small-scale enzymatic and other chemical reactions (Pang & Yeung, 2000). The sensitivity of these detection methods has been markedly improved in recent years such that the reaction volume used in many reactions is in excess of the minimum required for detection (Meldrum et al., 2000). Technical advances have allowed several of the previously mentioned methods to be performed in a high-throughput (e.g. thousands of reactions performed per day) and high-density (e.g. thousands of reaction performed on a surface of less than 100 square centimetres) manner (Shandrick et al., 2002). In addition, many of the components used in these reactions are very expensive (Azadan et al., 2002; Leung et al., 2000). Consequently, it is desirable to perform each reaction in the minimum volume required to enable detection, thus allowing further increases in throughput and density while reducing costs. Reaction volumes of less than one microlitre and within the nanolitre range have been found to be suitable for a number of detection technologies and reaction-formats (He et al., 2000; Pang & Yeung, 2000; Soper et al., 1998; Xue et al., 2001).

The examples below describe a method in which the addition of an inert liquid to an aqueous phase increases the overall volume of the system i.e. the inert liquid acts as a bulking agent. This allows for the volume of the aqueous phase comprising the reactants to be reduced below the level that can be handled by standard laboratory equipment. This inert phase consists of compounds that are not miscible with the aqueous phase. Such compounds are, typically, non-polar and are unable to interact with water and other polar compounds. Examples of such compounds are provided under the “Summary of the Invention” heading above.

In the examples provided below, the inert phase is chosen such that it does not interfere to any significant degree with the reaction that takes place in the aqueous phase. Non-interference is a function of the compound used and the chosen reaction to be performed. For example, the polymerase chain reaction (PCR) is not inhibited by the addition of mineral oil to the reaction. A mineral oil overlay is commonly used to prevent evaporation of the reaction during the DNA denaturation step (Saiki et al., 1988; Shimura et al., 2001).

In accordance with the present invention, and as described in the following examples, the discontinuous first phase and the continuous second phase are initially mixed such that a quasi-homogenous state is formed between the two phases before handling. Such a ‘liquid in a liquid’ colloidal state, consisting of two otherwise completely immiscible liquids, is often referred to as an emulsion (Schramm, 1993). Formation of emulsions between an aqueous solution and a non-polar substance can be facilitated and stabilised by the presence of detergents. Three general types of surfactants exist; the non-ionic surfactants, the ionic surfactants, and the zwitterionic surfactants (Helenius et al., 1979; Neugebauer, 1990). Examples of such surfactants have been included under the “Summary of the Invention” heading above.

In the examples that follow, an important property of the surfactants chosen is that their solubility in the aqueous phase and/or the inert phase can be changed, by either chemical or physical means, after formation of the emulsion. While solubility is an intrinsic property of each surfactant, it is also dependant on the composition of the phase it is dissolved in and the surrounding physical parameters (such as temperature or pressure). For example, a surfactant dissolved in water can be precipitated out (i.e. become insoluble) if high amounts of salt are added (Schott & Han, 1977; Schott & Royce, 1984). Furthermore, the solubility of many surfactants is dependent on the temperature. The temperature at which a particular surfactant becomes insoluble is known as its “cloud point” (Florence et al., 1975).

An important consideration in the choice of surfactant used to form the emulsions described in the examples, was that it should not interfere with the chemical or biochemical reaction performed in the aqueous phase. An example of a surfactant that is compatible with many enzymatic reactions is the non-ionic detergent TRITON X-100. This detergent is routinely added to the reaction buffers used in the polymerase chain reaction and is compatible with the activity of Taq DNA polymerase (eg. Taq DNA polymerase 10× reaction buffer (Promega, Wis., USA) contain 0.1% TRITON X-100). An additional consideration in the examples provided in the present application was that the detergent chosen should provide an emulsion of sufficient stability and viscosity such that it can be handled using standard fluid handling equipment.

The examples describe the formation of a stable emulsion between an aqueous and an inert phase that can be collapsed by changing the solubility of the surfactant in either phase. Changes to the solubility of the surfactant will cause a separation of the emulsified phases into two easily distinguishable volumes. For example, the emulsion formed between water and mineral oil in the presence of TRITON X-100 is stable at room temperature but collapses into an aqueous and an inert phase upon heating above 65° C.

In the examples, the emulsion is collapsed by a physical or chemical change to allow the aqueous phase to coalesce into a substantially continuous phase. In the emulsified state the aqueous phase is contained in a large number of small volumes surrounded by surfactants that are in turn surrounded by the inert phase. These small, dispersed volumes are often referred to as micelles. While enzymatic reactions have been performed within emulsions (Luisi et al., 1988; Walde et al., 1988), the efficiency of many reactions is less than that found in solely aqueous environments. For example, the processivity of the Klenow fragment of DNA polymerase I is greater in a purely aqueous phase than in a number of different emulsion systems (Anarbaev et al., 1998).

Collapsing an emulsion into two separate phases, and allowing the reaction to proceed in a solely aqueous environment achieves improvements in the efficiency of certain reactions. Furthermore, the collapse of the emulsion into two phases can allow the aqueous phase to interact with other components outside the emulsion. Interaction of the reaction phase with other components can be used to improve, inhibit, start, or stop the reaction. For example, if an emulsion containing a particular enzymatic substrate within the aqueous phase is added to a reaction vessel containing the enzyme then the substrate encapsulated in a micelle-structure will not be able to interact with the enzyme. Collapse of the emulsion provides access of the enzyme to the substrate allowing the reaction to occur. By this means a given reaction can be initiated by control of the conditions leading to emulsion collapse.

Different emulsions containing different phases can be mixed together without allowing the phases to interact. Upon collapse of the emulsions, the different reaction phases can interact, allowing control over reaction conditions and timing.

In the examples provided, a smaller aqueous volume is emulsified with a larger, inert volume. This emulsion can be handled with standard fluid handling tools (e.g. pipettes or robotic equipment). Upon performing the manipulation required to perform the reaction, a phase separation is induced by changing the solubility of the emulsifying surfactant. As a result a small aqueous phase has been manipulated without the use of specialised equipment. It is apparent that by maintaining a low ratio between the aqueous and inert phases that a small aqueous volume (e.g. less than a microlitre) can be handled as an emulsion of a larger volume (e.g. 10 microlitres or more).

The examples show that collapsible emulsions may be used in DNA sequencing reactions. The emulsions used in the examples significantly reduce the amount of reagents required for each DNA sequencing reaction and hence lead to a reduction of the overall cost of DNA sequencing. Ratios of aqueous to inert phases in the range of 1:4 to 1:19 have been successfully used. This allows DNA sequencing reactions to be performed in effective volumes of between two microlitres and 500 nanolitres using 10 microlitre emulsion volumes. It will be clear to those skilled in the art that other ratios and volumes are possible and that the ideal ratio and volume suitable for any particular application can be determined by simple trial and error.

The present invention seeks to overcome at least some of the difficulties presented in working with sub-microlitre reaction volumes. A key advantage of the invention is the avoidance of difficulties in pipetting and manipulating submicrolitre volumes. The invention can be used to avoid the need to invest in complicated and expensive technology such as capillary-based nanolitre-scale automated fluid handling systems (Meldrum, 2000), or nano-scale reactors for small-volume cycle sequencing reactions (He et al., 2000). The following examples demonstrate how this can be achieved.

### EXAMPLES

The following examples are offered by way of illustration and not by way of limitation. Unless defined otherwise, all technical and scientific terms used herein have the same meaning as commonly understood by one of ordinary skill in the art to which this invention belongs. Although any methods and materials equivalent to those described herein can be used in the practice or testing of the present invention, the preferred methods and materials are now described. All publications mentioned hereunder are incorporated herein by reference. Unless mentioned otherwise, the techniques employed or contemplated herein are standard methodologies well known to one of ordinary skill in the art and can be found in standard texts, such as that of Ausubel et al. (Ausubel et al., 1998).

Preferred embodiments of the invention will now be described by way of example only, with reference to the accompanying Figures.

### Example 1

### PCR Reactions in the Presence of TRITON X-100

This example demonstrates that the addition of TRITON X-100 has no significant effect on an enzyme reaction when added at levels required to create stable emulsions at 25° C. To establish that enzyme reactions can be performed efficiently in the presence of TRITON X-100, a simple titration experiment of increasing TRITON X-100 concentration in a PCR was performed. The PCR described below demonstrates that high concentrations of TRITON X-100 do not inhibit Taq DNA polymerase-based extension and amplification reactions.

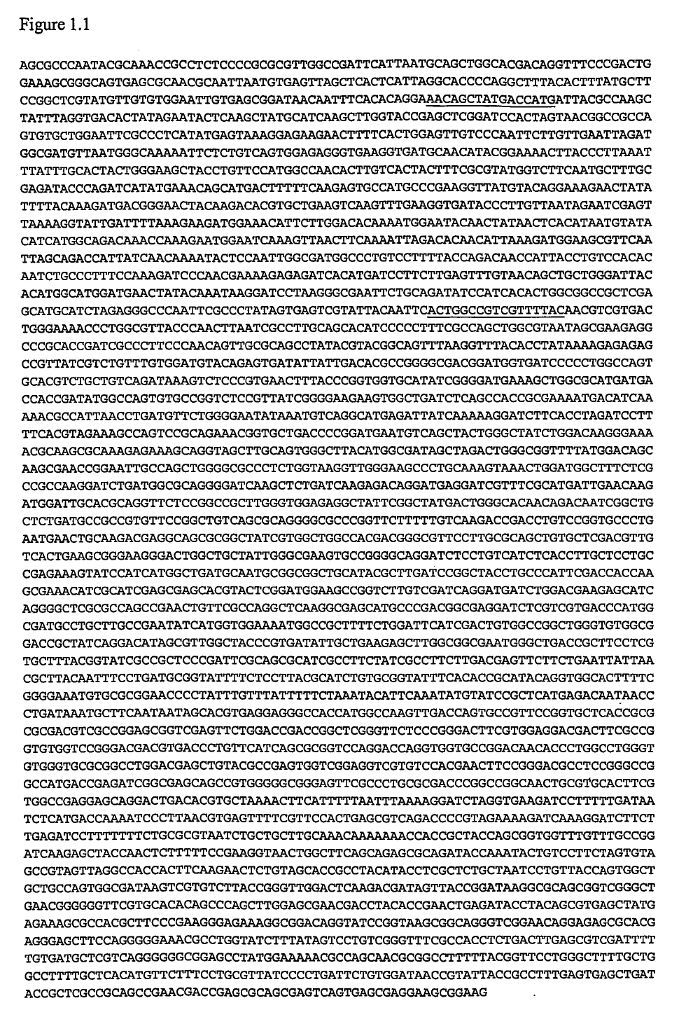

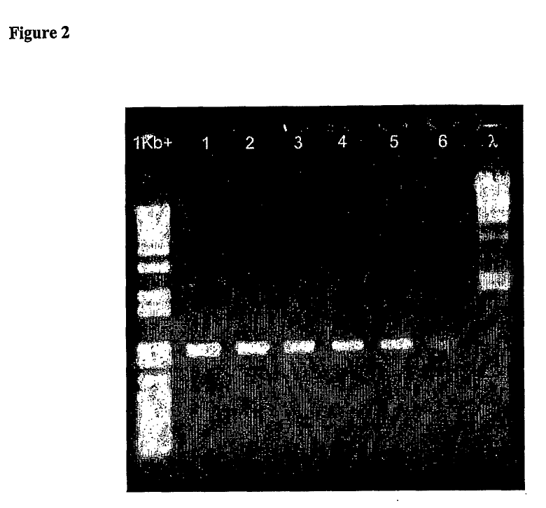

Each PCR contained the following components: 2 nanograms of the pCR-Blunt II-TOPO vector (Invitrogen, Carlsbad, Calif., USA) containing a 726 bp insert as template (FIG. 1), 5 pmol each of the M13 (−20A) forward sequencing primer (5′-ACTGGCCGTCGTTTTAC-3′; SEQ ID No. 6) and M13 (−21) reverse sequencing primers (5′-AACAGCTATGACCATG-3′; SEQ ID No. 7), 2 microlitres of 25 mM MgCl sub. 2, 2 microlitres of 2 mM dNTPs, 2 microlitres of 10× PCR buffer (Thermophilic DNA polymerase, magnesium free buffer; Promega, Madison, Wis., USA), 1 unit of Taq DNA polymerase (Promega), 0.1 unit of Pfu DNA polymerase (Promega), and water to final volume of 20 microlitres. To six separate PCR reactions either 0, 0.5, 1, 2, 4, or 8% (vol:vol) of TRITON X-100 was added. The PCRs were performed in 0.2 ml thin-walled tubes using the following conditions: 2 min at 96° C., 20 cycles of 95° C. for 30 s, 50° C. for 30 s, and 72° C. for 1 min, followed by a 72° C. hold for 5 min. The reactions were stored at 4° C. Two microlitres of each PCR reaction were run on a 1% (w/vol) agarose gel in 1× TAE-buffer (40 mM Tris-acetate, 1 mM EDTA) before being stained with ethidium bromide and visualised under UV irradiation (FIG. 2).

The experiment shows that the addition of TRITON X-100 at a concentration of 0.5, 1 and 2% had no apparent effect on PCR amplification (FIG. 2; lanes 2, 3 & 4). At a concentration of 4% the TRITON X-100 had a small effect, while at 8% the efficiency of amplification was significantly reduced. This result demonstrates that concentrations of TRITON X-100 below 2% had no apparent effect on the PCR.

### Example 2

### Temperature-Induced Collapse of Emulsions

Typically, it will be required that the emulsion formed remains relatively stable during reaction set-up and that it collapses just prior to, or during, the reaction process. One means of accomplishing this requirement is by creating the emulsion at a temperature at which it is stable and then performing an incubation step at a higher temperature at which the emulsion is unstable and collapses. The temperature at which the thermal-induced collapse of an emulsion occurs is a function of the inherent cloud point of the surfactant, the concentration of the surfactant, and the presence of additives that affect the solubility of the surfactant either in the aqueous or organic phases (Gu & Galera-Gomez, 1999).

Therma-Induced Collapse of Triton-X100 or Triton-X114 Emulsions with Mineral Oil

An experiment was performed to establish the temperature profile of different emulsions using incubations at stepwise temperature increments. Emulsions were created consisting of 20 microlitres of either a 0.5%, 1.5% or 3% of TRITON X-100 or TRITON X-114 in water and 180 microlitres of light mineral oil (Sigma-Aldrich). The emulsions were prepared in 2 mL microcentrifuge tubes (Quantum Scientific, Paddington, Australia) by intermittent vortexing using a bench top vortex mixer (Model VM1; Ratek Instruments Pty Ltd., Boronia, Australia) at the maximum setting. Upon formation of a visible emulsion, the tubes were vortexed for an additional 2 min. All manipulations were performed at room temperature (˜25° C.). To assist in the creation of the 0.5% TRITON X-100 and TRITON X-114 emulsions approximately 50 mg of 0.1 mm diameter glass beads were added to each tube to increase the effective mechanical agitation during vortexing.

The emulsions were incubated at 37° C., 45° C., 55° C., 65° C., 85° C., 95° C. for 10 min and the stability of the emulsion assessed visually. This process was performed in a stepwise fashion starting at 37° C. After each temperature incubation the samples were removed from the incubation block and the temperature of the heating block raised to the next temperature. The samples were then returned to the heating block and incubated for 10 min. This process was repeated until the final 95° C. incubation was performed. The estimated proportion of the emulsion collapse at each temperature is shown in Table 2.1.

The cloud points of 1% aqueous solutions of TRITON X-100 or TRITON X-114 are 63 to 69° C. and 20° C. to 22° C., respectively (Product information sheet, Sigma-Aldrich, St. Louis, USA). As shown in Table 2.1, the TRITON X-100 based emulsions are more thermally stable than the comparable (same percentage) TRITON X-114 emulsions. The temperature at which a given emulsion collapses rises proportionally with the concentration of the surfactant. For example, as the concentrations of TRITON X-100 is increased from 0.5%, to 1.5% and 3.0%, the temperature at which the emulsion collapses rises from 45-55° C., to 65-85° C., and greater than 95° C., respectively. Similarly, as the concentrations of TRITON X-114 is increased from 0.5%, 1.5% and 3.0%, the temperature at which the emulsion collapses rises from 37-45° C., to 45-55° C., and 65-85° C., respectively.

This example demonstrates that the temperature range at which emulsion collapse occurs can be manipulated by adjusting the surfactant concentration.

TABLE 2.1
Thermal-induced collapse of emulsions with various concentrations of
TRITON X-100 or TRITON X-114 and mineral oil.

| Temp | Triton-X 100 | | | Triton-X 114 | | |
|---|---|---|---|---|---|---|
| ° C. | 0.5% | 1.5% | 3.0% | 0.5% | 1.5% | 3.0% |
| 37 | intact^{a} | intact | intact | intact | intact | intact |
| 45 | Intact | intact | intact | ˜40 | intact | intact |
| 55 | ˜80^{b} | intact | intact | ˜20 | ˜80 | intact |
| 65 | ˜80 | intact | intact | ˜20 | ˜40 | intact |
| 85 | ˜40 | ˜40 | intact | ˜20 | ˜20 | ˜40 |
| 95 | ˜30 | ˜30 | intact | ˜20 | ˜20 | ˜40 |

^{a}Emulsion remains homogeneous without sign of collapse.
^{b}Estimated percentage of the emulsion that remains intact.

Thermal-Induced Collapse of Triton-X100 or Triton-X114 Emulsions Made with Various Mixtures of Mineral Oil and Dodecane.

An experiment was performed to establish the temperature stability of emulsions consisting of 50 microlitres of either 1.5% TRITON X-100 or TRITON X-114 in water and 450 microlitre mixtures of mineral oil and dodecane at ratios of 450/0, 425/25, 400/50, 350/100, 300/150, 250/200 or 0/450, respectively. The emulsions were created as described in the previous example.

The emulsions were first incubated in a water bath at 37° C. for 30 min so that the initial stability could be assessed. The samples were placed in a bench-top centrifuge and spun for a short pulse of five seconds prior to assessment. The degree to which each emulsion collapsed at each temperature is shown in Table 2.2. The samples were re-vortexed to form an emulsion prior to the next incubation at the higher temperature. The temperature of the water bath was equilibrated at the next temperature prior to the next incubation.

The TRITON X-100 based emulsions proved more thermally stable than the comparable (same percentage) TRITON X-114 emulsions (Table 2.2). The data clearly demonstrates that the thermal stability of each emulsion is affected by the ratio of mineral oil:dodecane used (Table 2.2). The TRITON X-100 and TRITON X-114 emulsions in mineral oil (mineral oil:dodecane ratio of 450/0) are the most stable and give rise to emulsions that collapse at temperatures greater than 90° C., and at the temperature range of 60-75° C., respectively. Increasing the amount of dodecane in the inert phase, at ratios of 425/25, 400/50 and 350/100, had no discernible effect on the temperature at which the emulsion collapsed. Higher levels of dodecane in the TRITON X-100 emulsion at ratios of 300/150, 250/200 and 0/450 gave rise to emulsions that collapse at a temperature range of 65-75° C. This represents a lowering of the thermal stability of the TRITON X-100 emulsions by approximately 15° C. Higher levels of dodecane in the TRITON X-114 emulsions at ratios of 300/150, 250/200 and 0/450 gave rise to emulsions that collapse at a temperature range of 50-60° C. Similarly, this represents a lowering of the thermal stability of the TRITON X-114 emulsions by approximately 10° C.

Table 2.2 Thermal induced collapse of emulsion with 1.5% TRITON X-100 (A) or TRITON X-114 (B) using various mineral oil and dodecane mixtures.

A. TRITON X-100

| Temp. | Ratio of mineral oil:dodecane (vol:vol) | | | | | | |
|---|---|---|---|---|---|---|---|
| ° C. | 450/0 | 425/25 | 400/50 | 350/100 | 300/150 | 250/200 | 0/450 |
| 42 | intact^{a} | intact | intact | intact | intact | intact | intact |
| 50 | intact | intact | intact | intact | intact | intact | intact |
| 60 | intact | intact | intact | intact | intact | intact | intact |
| 75 | intact | intact | intact | intact | ˜80^{b} | ˜70 | ˜70 |
| 90 | intact | intact | intact | intact | ˜80 | ˜70 | ˜60 |

B. TRITON X-114

| Temp. | Ratio of mineral oil:dodecane (vol:vol) | | | | | | |
|---|---|---|---|---|---|---|---|
| ° C. | 450/0 | 425/25 | 400/50 | 350/100 | 300/150 | 250/200 | 0/450 |
| 42 | intact | intact | intact | intact | intact | intact | intact |
| 50 | intact | intact | intact | intact | intact | intact | intact |
| 60 | intact | intact | intact | intact | ˜80 | ˜80 | ˜80 |
| 75 | ˜20 | ˜20 | ˜20 | ˜20 | ˜20 | ˜20 | ˜20 |
| 90 | ˜20 | ˜20 | ˜20 | ˜20 | ˜20 | ˜20 | ˜20 |

^{a}Emulsion remains homogeneous without sign of collapse.
^{b}Estimated percentage of the emulsion that remains intact.

This example demonstrates that the temperature range at which an emulsion collapses can be manipulated by changing the nature of the inert phase. In parallel to the decrease in the thermal stability of the emulsions with a high proportion of dodecane, it was noticed that these emulsions had better properties with regard to handling, whereby pipetting of the emulsion was easier.

### Example 3

### Change of Reaction Conditions Through Collapse of an Emulsion

This example demonstrates that an emulsion phase separation (collapse) can be used to change the reaction conditions within the aqueous phase. The emulsion was created from an aqueous solution of the DNA staining compound ethidium bromide in the non polar solvent hexadecane using the detergent TRITON X-114. Ethidium bromide interacts with DNA by intercalation resulting in strong fluorescence under UV-light in the presence of DNA. The emulsion was added to a tube containing dried DNA on its inner surface. Only upon collapse of the emulsion will the DNA and ethidium bromide be able to interact and fluoresces.

The emulsion was prepared in a 2 ml microcentrifuge tube (Product number: 508-GRN; Quantum Scientific, Paddington, Australia) by vortexing using the VM1 vortex mixer (Ratek Instruments Pty Ltd., Boronia, Australia) at maximum setting. Forty microlitres of water was mixed with 10 microlitres of 10% (v/v) TRITON X-114 (Sigma). Nine hundred microlitres of hexadecane (Sigma) was added in small amounts and with continuous vortexing of the sample. Care was taken to ensure that before each new addition of hexadecane the mixture had formed a homogenous emulsion. One hundred and eight microlitres of this emulsion were then mixed with 9.5 microlitres of TE-buffer (10 mM Tris-HCl/1 mM EDTA, pH8) and 0.5 microlitres of ethidium bromide (10 micrograms per millilitre). This emulsion is referred to as the “EtBr-emulsion” hereafter.

Reaction tubes were prepared with dried DNA by adding 1 microlitre (1 microgram per microlitre) of 1 Kb plus DNA ladder (Invitrogen, Carlsbad, Calif., USA) to the bottom of the tube and drying at 80° C. to completion. In a control reaction 1 microlitre of water was used instead of the DNA.

Fifty microlitres of EtBr-emulsion was added to each reaction tube before heating for 2 min at 95° C. to induce phase separation. After heating, the aqueous phase was found in the bottom of the tube and was overlain by the non-polar hexadecane phase. A control reaction was performed that was not heated (i.e. phase separation was not induced). The tubes were then exposed to UV-light and photographed using the GelDoc System (Bio-Rad, Hercules, Calif., USA).

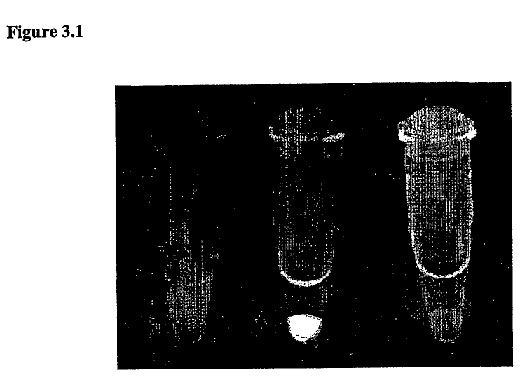

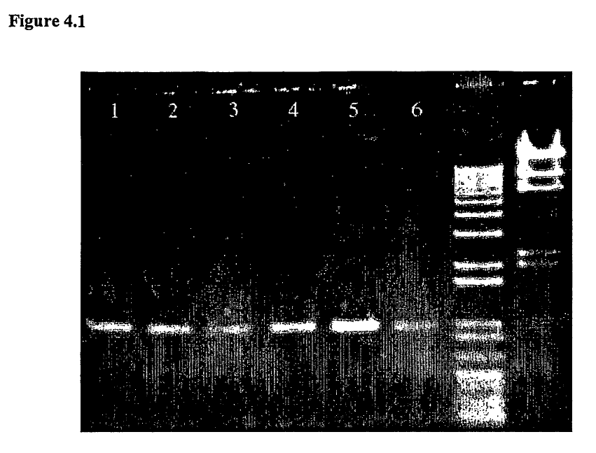

FIG. 3 shows that the ethidium bromide within an intact emulsion is not able to interact with the DNA contained on the tube wall, as no fluorescence was observed (left tube, FIG. 3). Upon collapse of the emulsion, the aqueous phase is able to come into contact with the DNA and as a consequence fluorescence is observed (middle tube, FIG. 3). This fluorescent signal is not simply due to a change in volume of the aqueous phase (micelles in intact emulsion versus coalesced phase in the collapsed emulsion), because the collapsed EtBr-emulsion in the absence of DNA shows no fluorescence (right tube, FIG. 3).

In conclusion, this example illustrates that the collapse of an emulsion can be used to initiate a chemical reaction (i.e. the chemical staining of DNA).

### Example 4

### Demonstration of PCR in Collapsible Emulsions

This example demonstrates that an enzymatic reaction can be performed in a collapsible emulsion. A PCR was performed and the initial high temperature denaturation steps of the thermal cycling procedure allowed efficient separation of the emulsion's two phases.

Six PCRs were performed containing either a 0, 0.5, 1, 2, 4, or 8% final aqueous concentration of TRITON X-100. Each PCR mix contained: 1 nanogram of TOPO plasmid DNA template (as described in Example 1), 2.5 pmol each of the M13 outer forward (−47) (5′-CGCCAGGGTTTTCCCAGTCACGAC-3′; SEQ ID No. 8) and outer reverse (−48) primers (5′-AGCGGATAACAATTTCACACAGGA-3′; SEQ ID No. 9), 1 microlitre of 25 mM MgCl sub. 2, 1 microlitre of 2 mM dNTPs, 1 microlitre of 10× Taq buffer (Thermophilic DNA polymerase, magnesium free buffer; Promega), either 0, 0.5, 1, 2, of 10% (vol:vol) TRITON X-100, or 1.6, or 3.2 microlitres of 25% (vol:vol) TRITON X-100, and water to a final volume of 8.5 microlitre.

To the TRITON X-100-free control, 0.5 units of Taq DNA polymerase (Promega) and 0.05U Pfu DNA polymerase (Promega) was added in a total of 1.5 microlitres of water to give a final volume of 10 microlitres. The positive control was performed in duplicate before combining to give a single 20 microlitre reaction volume.

To each PCR containing TRITON X-100, 90 microlitres of mineral oil was added in 30 microlitres aliquots and an emulsion created by vortexing the sample. To each of these emulsions 0.5 units of Taq DNA polymerase (Promega) and 0.05 units of Pfu DNA polymerase (Promega) in 1.5 microlitres of water was added. The reactions were vortexed for 3 sec.

Twenty microlitre aliquots were transferred from each emulsion into 0.2 ml thin walled PCR tubes, as was the entire positive control, and subjected to the following thermal cycling conditions: 2 min at 96° C., then 20 cycles of 95° C. for 30 s, 50° C. for 30 s and 72° C. for 1 min, followed by a 72° C. hold for 5 min. The reaction was stored at 4° C.

The oil was removed from the PCR reactions by extracting twice with 200 microlitres water-saturated ether. Two microlitre of each PCR reaction were run on a 1% (w/v) agarose gel with TAE buffer, stained with ethidium bromide and visualised under UV irradiation. The resulting gel image is shown in FIG. 4.

This example demonstrates that PCR amplification can occur efficiently in reaction samples made from collapsible emulsions of mineral oil using varying TRITON X-100 concentrations.

### Example 5

### Demonstration of Cycle DNA Sequencing in Collapsible Emulsions

Cycle DNA sequencing, like PCR, is well suited to collapsible emulsion-based reaction mixes since the initial high temperature denaturation steps of the thermal cycling procedure can be used to simultaneously efficiently collapse the emulsion. Collapsible emulsion-based DNA sequencing allows for substantial reductions in the volume of materials required and hence the cost of expensive fluorescent dye-terminator premix.

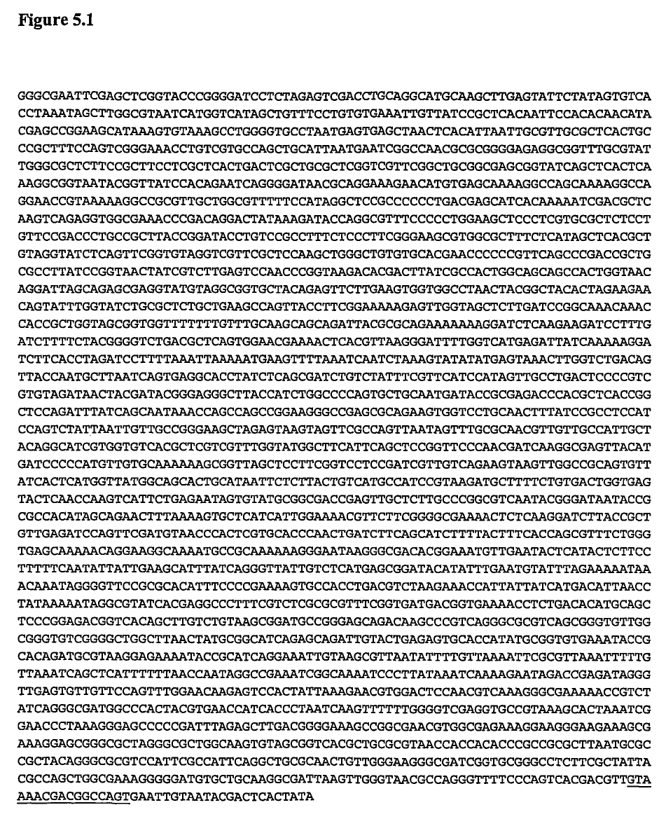

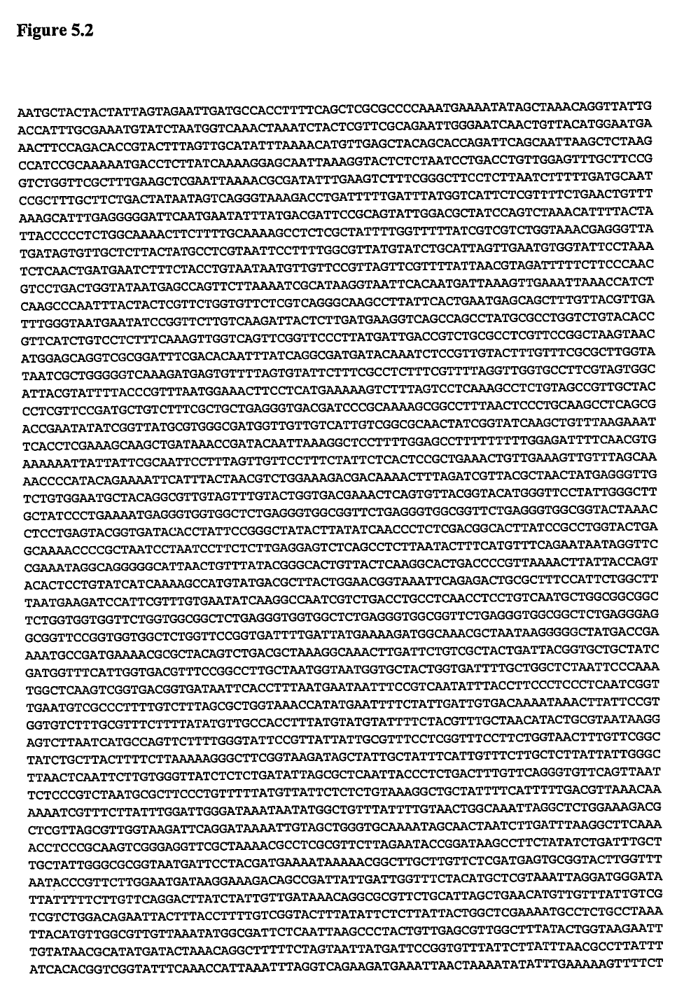

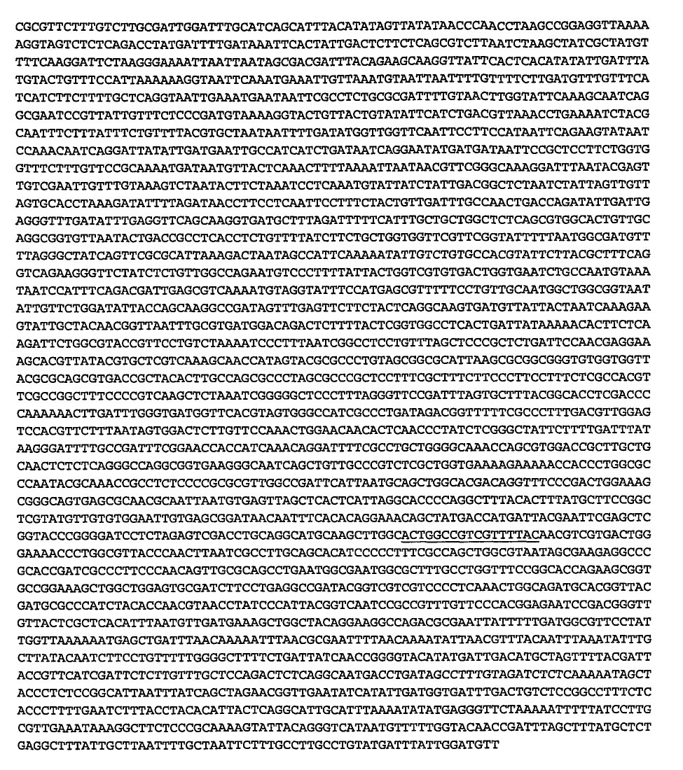

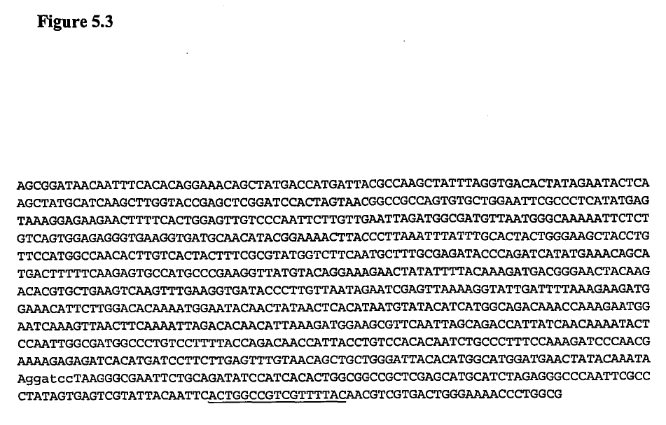

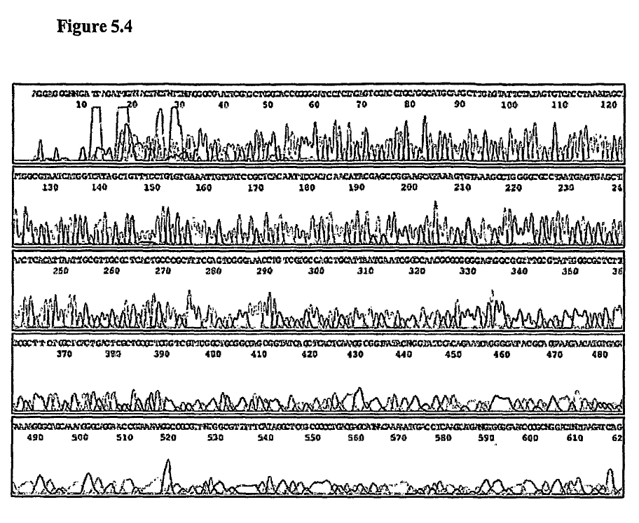

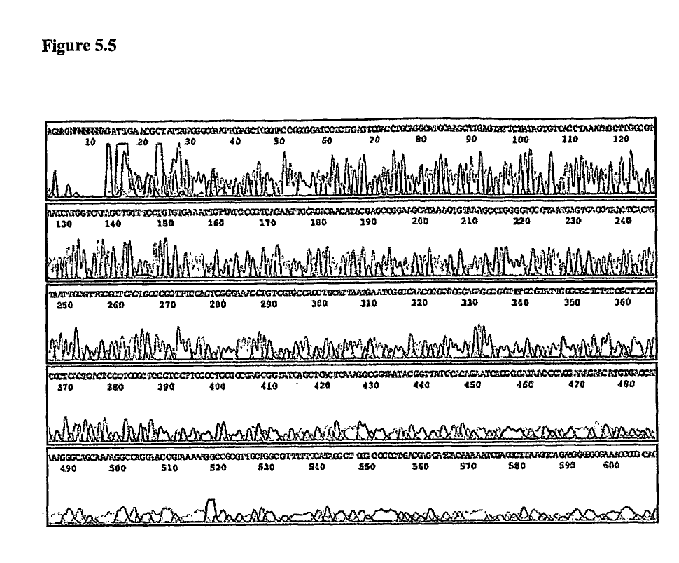

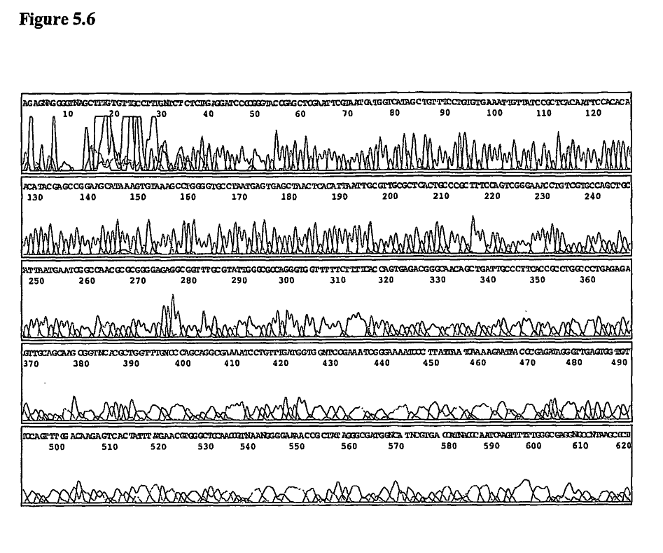

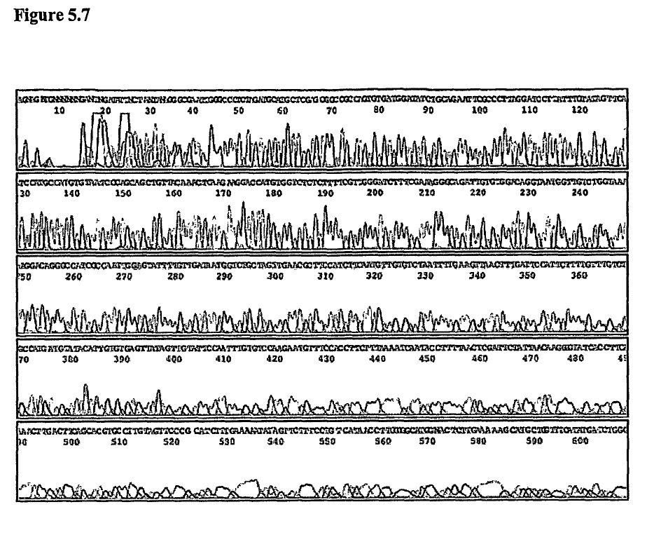

Three DNA templates were sequenced using emulsion-based sequencing reactions: the double stranded plasmid pGEM3Zf(+) (GenBank/EMBL Accession Number X65306; SEQ ID No. 2; FIG. 5.1), the single stranded M13pm18(+)(GenBank/EMBL Accession Number X02513; SEQ ID No. 3; FIG. 5.2), and a 1016 base pair PCR product (SEQ ID No. 4; FIG. 5.3). The pGEM and M13 DNA templates were obtained from Amersham Biosciences. The 1016 bp PCR was performed as described in Example 1. To clean the reaction for sequencing, 0.25 units of exonuclease I (New England Biolabs) and 0.25 units of arctic shrimp alkaline phosphatase were added. The reaction was incubated at 37° C. for 30 min followed by a heat denaturation step at 95° C. to inactivate the enzymes.

Each of the sequencing reactions contained either 100 ng of pGEM3Zf(+), 10 ng of PCR product, or 20 ng of M13pm18 (+). The three DNA templates were added to an empty 0.2 ml thin-walled PCR reaction tube and dried by heating the tubes to 80° C. for 10 min before the sequencing emulsion was added.

The emulsion was prepared in a 2 ml microcentrifuge tube (Product number: 508-GRN; Quantum Scientific, Paddington/Australia) with vortexing being performed using the VM1 mixer (Ratek Instruments Pty Ltd., Boronia/Australia) at maximum setting. Each emulsion contained: 40 pmol of M13 forward sequencing (−20B) primer (5′-GTAAAACGACGGCCAG-3′; SEQ ID No. 10), 1.6 microlitres of a 10% (vol:vol) TRITON X-100, and water to give a final volume of 12 microlitres. To this mixture, 180 microlitres of mineral oil was added in 60 microlitres aliquots. The sample was vortexed after each addition of oil to give a final emulsion volume of 192 microlitres. To this emulsion of 8 microlitres of BigDye Version 2 fluorescent dye-terminator premix (Applied Biosystems) was added and briefly vortexed for three seconds. The order of reagent addition was designed to minimise agitation that may denature the sensitive components (i.e. the DNA polymerase). Immediately, 10 microlitre aliquots were taken from this emulsion and added to the 0.2 ml reaction tubes containing the dry DNA templates—the DNA templates can migrate to the aqueous phase over time and, depending on the conditions, this may occur before, during and/or after the collapse of the emulsion.

A positive control non-emulsion-based DNA sequencing reaction was performed containing: 5 pmol of M13 forward sequencing (−20) primer (5′-GTAAAACGACGGCCAG-3′; SEQ ID No. 10), 1 microlitre of BigDye™ Version 2 (Applied Biosystems), 3 microlitres of 2.5× dilution buffer (200 mM Tris-HCl, pH 9.0; 5 mM MgCl sub.2), 0.8 microlitres of 10% (vol:vol) TRITON X-100, and water to a final volume of 10 microlitres. This mix was added to 1 microgram of dry pGEM3Z(f+) template DNA in 0.2 ml PCR tube.

All sequencing reactions were performed using a 9700 thermal cycler (Applied Biosystems) using the following conditions: 96° C. for 30 s, 40 cycles of 96° C. for 10 s, 50° C. for 5 s, and 60° C. for 4 min.

The sequencing reactions were transferred to 1.5 ml centrifuge tubes containing 40 microlitres of water and 40 microlitres of Tris-buffered phenol (Sigma-Aldrich). The samples were vortexed for 5 s before centrifugation at 14,000 g for three min in a bench top microcentrifuge. The top layer aqueous phases were transferred to fresh tubes and the DNA precipitated by addition of nine volumes of n-butanol, vortexing for 10 s, and centrifugation at 14,000 g for 15 min. The supernatants were discarded and the residual butanol removed by evaporation at 80° C. for 10 min. The dry, dye-labelled, DNA products of each sequencing reaction were dissolved in two microlitres of standard gel loading buffer according to the manufacturer's instructions and 0.6 microlitres loaded onto the gel. The electropherogram of the control DNA sequencing reaction is shown in FIG. 5.4. Electropherograms of the DNA sequencing reaction for the pGEM3Zf(+), the M13mp18(+) and 1016 bp PCR product templates are shown in FIGS. 5.5, 5.6 and 5.7.

These data clearly demonstrate that the emulsion-based sequencing reactions can be performed in collapsed emulsion and that the mineral oil and TRITON X-100 at a concentration of 0.8% have no observable inhibitory effect on the efficiency of the reaction.

These results also demonstrate that one microlitre DNA sequencing reactions (10 microlitre emulsion) can be performed using standard liquid handling equipment.

### Example 6

### DNA Sequencing in Various Collapsible Emulsions

This example demonstrates that DNA sequencing reactions can be performed in a variety of collapsible emulsions. The emulsions were created from different organic phases and different surfactants. Three different emulsions were created: a hexadecane/TRITON X-114-based emulsion, a mineral oil/octanol/TRITON X-114-based emulsion, and a mineral oil/octanol/TRITON X-100-based emulsion.

Hexadecane/TRITON X-114-Based Emulsion

The emulsion was prepared in a 2 ml microcentrifuge tube (Product number: 508-GRN; Quantum Scientific, Paddington, Australia) with vortexing on a VM1 vortex mixer (Ratek Instruments Pty Ltd., Boronia, Australia) at maximum setting. Forty microlitres of water were mixed with 10 microlitres of 10% (vol:vol) TRITON X-114 (Sigma-Aldrich). Nine hundred microlitres of hexadecane (Sigma-Aldrich) was added in small amounts (approximately 100 microlitres at a time) with continuous vortexing of the sample. Care was taken to ensure that before each new addition of hexadecane the mixture had formed a homogenous emulsion.

Mineral Oil/Octanol/TRITON X-114-Based Emulsion

The emulsion was prepared in a 2 ml microcentrifuge tube (Quantum Scientific) with vortexing on a VM1 vortex mixer (Ratek Instruments) at maximum setting. Forty microlitres of water was mixed with 10 microlitres of 10% (vol:vol) TRITON X-114 (Sigma-Aldrich). Eight hundred and forty microlitres of mineral oil (Sigma-Aldrich) was added in small amounts (approximately 100 microlitres at a time) with continuous vortexing of the sample. Care was taken to ensure that before each new addition of mineral oil the mixture had formed a homogenous emulsion. Sixty microlitres of n-octanol (Sigma-Aldrich) was added and the emulsion vortexed for 5 min.

Mineral Oil/Octanol/TRITON X-1100-Based Emulsion

The emulsion was prepared in a 2 ml microcentrifuge tube (Quantum Scientific) with vortexing on a VM1 vortex mixer (Ratek Instruments) at maximum setting. Forty microlitres of water was mixed with 10 microlitres of 10% (vol:vol) TRITON X-100 (Sigma-Aldrich). Eight hundred microlitres of mineral oil (Sigma) was added in small amounts (approximately 100 microlitres at a time) with continuous vortexing of the sample. Care was taken to ensure that before each new addition of mineral oil, the mixture had formed a homogenous emulsion. One hundred microlitres of n-octanol (Sigma) was added and the emulsion vortexed for 5 min.

All emulsions were stored at room temperature and vortexed for 30 sec before use.

Emulsion-Based DNA Sequencing Reactions

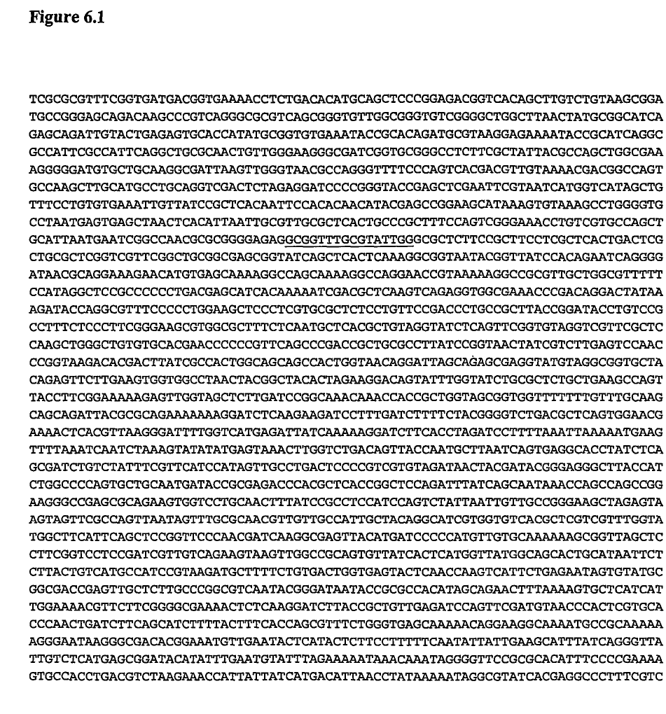

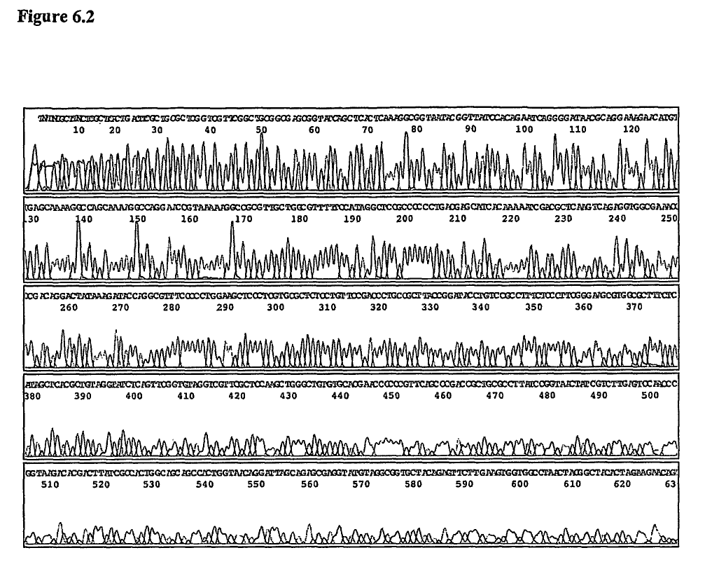

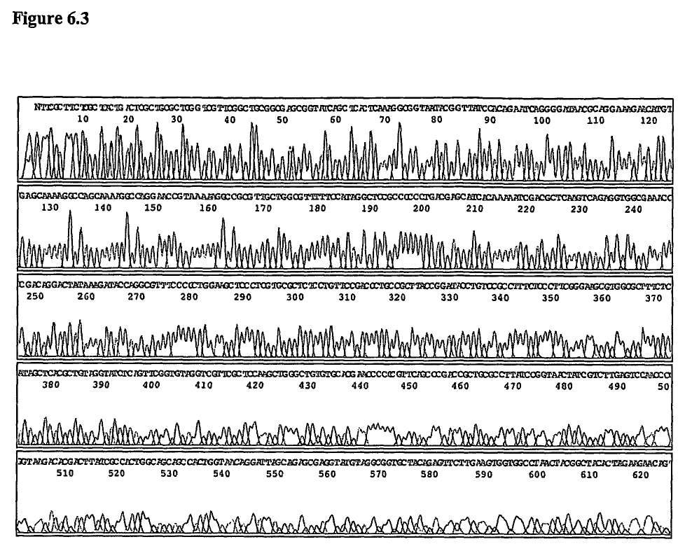

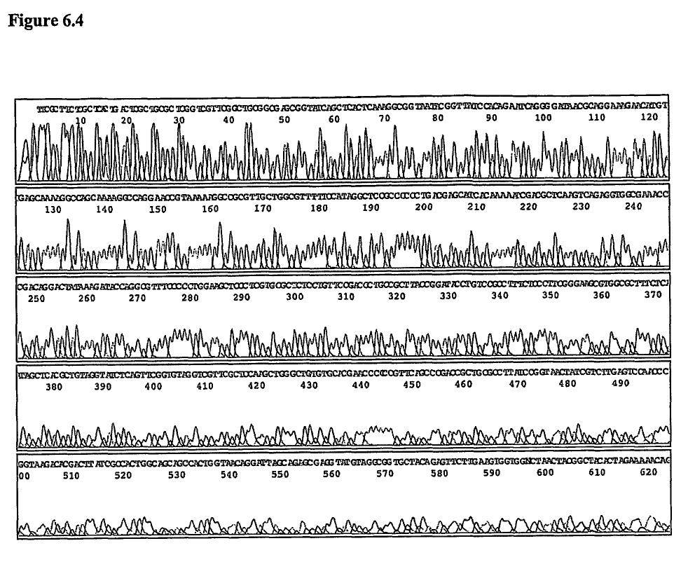

The DNA templates were prepared by adding 200 ng of HindIII linearized pUC18 plasmid DNA to a 0.2 ml PCR tube. The DNA was dried down at 80° C. for 30 min. The sequence of pUC 18 and the binding site for the primer pGemEcoRV are shown in FIG. 6.1.

Forty-seven and a half microlitres of each emulsion were aliquoted into 1.5 ml microcentrifuge tubes (Product number 72.690; Sarstedt, Adelaide, Australia). To each emulsion 25 pmol of the pGemEcoRV primer (5′-ATCGCGGTTTGCGTATTGG-3′; SEQ ID No. 11) and 2 microlitres of BigDye™ Version 3 (Applied Biosystems) premix were added. The emulsions were mixed by repetitive pipetting (10-20 times) and vortexing for 1 s. Ten microlitres of each emulsion (containing primer and BigDye premix) were added to the 0.2 ml PCR tubes with dried down template DNA. The reactions were heated to 96° C. for 3 min before being cycled 40 times at 96° C. for 10 s, 45° C. for 30 s, and 60° C. for 4 min.

Excess labelled nucleotides and salts were removed from the reaction by adding 100 microlitres of water to each reaction and transferring the entire mixture to a fresh centrifuge tube containing 100 microlitres of phenol. The mixtures were vortexed for 5 s and centrifuged for 5 min at 14,000 g. The aqueous phases were transferred to fresh tubes containing 900 microlitres of n-butanol. The water/butanol mixtures were vortexed for 10 s and centrifuged for 10 min at 14,000 g. The supernatants were discarded and the pellets dried for 5 to 10 min at 80° C. The pellets were resuspended in four microlitres loading dye (Applied Biosystems) before 2.5 microlitres were loaded onto a ABI 3700 capillary DNA sequencer according to the manufacturer's instructions (Applied Biosystems).

Good sequence data of the correct sequence was obtained for all three emulsions tested. FIGS. 6.2, 6.3 and 6.4 show the electropherograms for the hexadecane/triton X-114 emulsion, the mineral oil/octanol/triton X-114 emulsion and the mineral oil/octanol/triton X-100 emulsion, respectively. These results demonstrate that DNA sequencing reactions can be successful performed using a range of different collapsible emulsions.

### Example 7

### Comparison of collapsible Emulsion DNA Sequencing with Diluted Premix DNA Sequencing

This example compares the success and efficiency of DNA sequencing performed using two different approaches to reduce the usage of sequencing reagent premix. The first system (hereafter termed “diluted premix”) consists of a diluted reaction where the amount of sequencing chemistry premix is reduced while the total aqueous reaction volume is kept constant. In the second system (hereafter termed “collapsible emulsion”) the reaction utilises an undiluted premix and the reaction occurs in a collapsed emulsion. As such the aqueous reaction volume, and hence the amount of premix used, is reduced.

The diluted premix reactions contained 0.4 microlitre of BigDye Version 3 premix (Applied Biosystems), 3.6 microlitres of dilution buffer (200 mM Tis-HCl, pH9; 5 mM magnesium chloride), 5 pmol of pGemEcoRV primer (5′-ATCGCGGTTTGCGTATTGG-3′; SEQ ID No. 11), and water to 10 microlitres. This solution was transferred to a tube containing 200 ng of dried pUC18 plasmid DNA template prepared as described in Example 6.

For the collapsible emulsion system an emulsion was prepared in a 2 ml microcentrifuge tube (Quantum Scientific) with vortexing on a VM1 vortex mixer (Ratek Instruments) at maximum setting. Forty microlitres of water was mixed with 10 microlitres of 10% (vol:vol) TRITON X-100 (Sigma). Nine hundred microlitres of mineral oil (Sigma) was added in small amounts with continuous vortexing of the sample. Care was taken to ensure that before each new addition of mineral oil the mixture had formed a homogenous emulsion. Forty-seven and a half microlitres of this emulsion was aliquoted into 1.5 ml microcentrifuge tubes (Product number 72.690; Sarstedt, Adelaide/Australia). To the emulsion 25 pmol of pGemEcoRV primer and 2 microlitres of BigDye Version 3 premix (Applied Biosystems) was added and mixed by repetitive pipetting (10-20 times) and vortexing for 1 sec. Ten microlitres of the emulsion was added to a reaction tube containing 200 ng of dried pUC18 plasmid DNA template prepared as described in Example 6.

Both reactions (diluted premix and collapsible emulsion) were heated for 3 min at 96° C. before being cycled 40 times at 96° C. for 10 s, 45° C. for 30 s and 60° C. for 4 min. Excess nucleotides were removed and the samples analysed as described in Example 6.

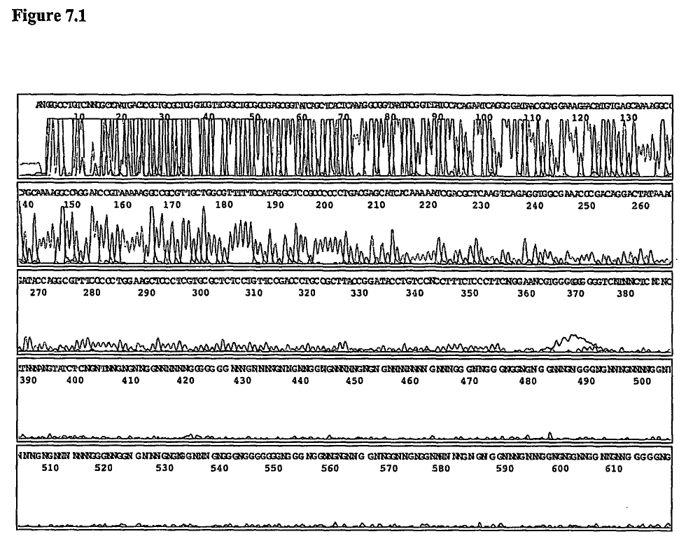

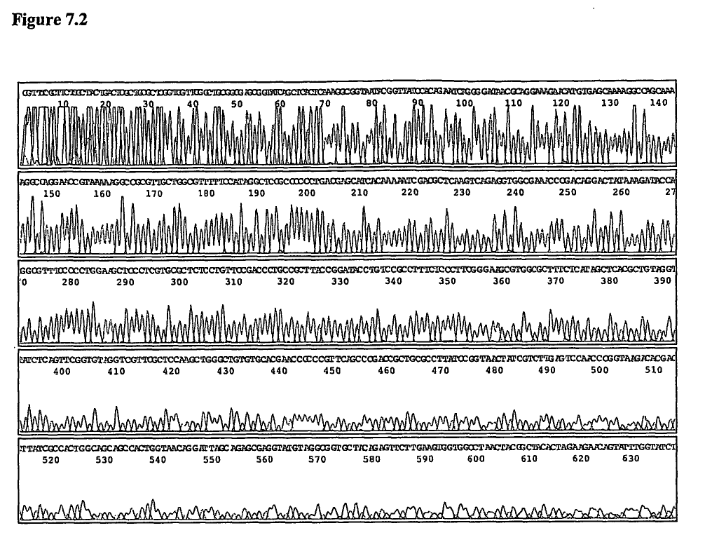

The diluted premix reaction (FIG. 7.1) provided low quality sequence data with a short read length (e.g. beyond approximately position 350 the sequence is unreadable). In contrast, the collapsible emulsion reaction provided high quality sequence data with readable data beyond position 600 FIG. 7.2).

This example demonstrates that the success and efficiency of a sequencing reaction with fixed amounts of sequencing chemistry performed in the small-aqueous volume provided by a collapsible emulsion is greater than in a relatively larger and diluted aqueous volume.

### Example 8

### Removal of the Inert Phase of a Collapsed Emulsion

This example demonstrates that the inert phase can be removed from an aqueous phase after an emulsion of the two phases has been collapsed. The emulsion was created from an aqueous phase and the non-polar solvent n-hexane using the detergent TRITON X-114. After temperature-induced collapse of the emulsion, the hexane phase is removed by evaporation.

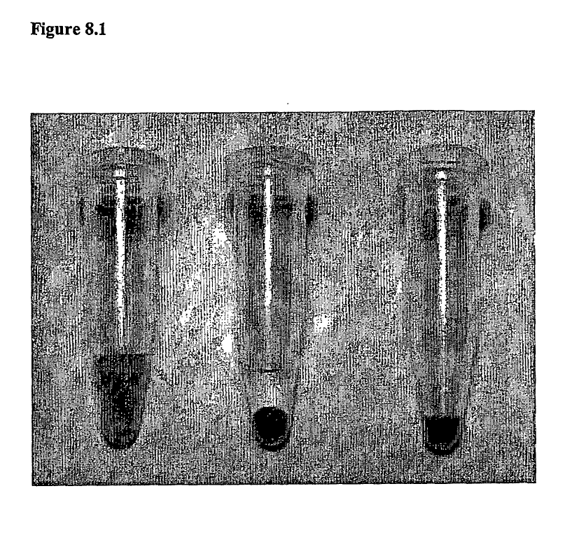

The emulsion was prepared in a 2 ml microcentrifuge tube (Product number: 508-GRN; Quantum Scientific, Paddington, Australia) by vortexing using the VM1 vortex mixer (Ratek Instruments Pty Ltd., Boronia, Australia) at maximum setting. Forty-five microlitres of water was mixed with 5 microlitres of 10% (v/v) TRITON X-114 (Sigma). Nine hundred microlitres of n-hexane (Sigma-Aldrich) was added in small amounts (approximately 100 microlitres at a time) with continuous vortexing of the sample. Care was taken to ensure that before each new addition of hexane the mixture had formed a homogenous emulsion. Ninety-five microlitres of this emulsion were then mixed in a 1.5 ml microcentrifuge tube with 5 microlitres water containing 2 micrograms per millilitre of Cresol Red (Sigma-Aldrich). Cresol Red is an pH indicator dye and was used to specifically colour the aqueous phase. Phase separation was induced by heating the emulsion for 5 min at 95° C. The collapsed emulsion was then centrifuged for 2 min at 20 000× g. After this the hexane-phase was evaporated by placing the tube into a DNA110 Speed Vac (Savant, Waltham, Mass., USA) at medium heat-setting for approximately 3 min. FIG. 8 shows the progress of this experiment.

This example demonstrates that the inert phase can be removed from the aqueous phase after the emulsion has been collapsed and, further, that evaporation of the inert phase may be used as a means of removing the inert phase. Clearly, other methods of removing the inert phase could also be used, including physical removal of the inert phase by, for example, careful pipetting.

### Example 9

### Collapse of an Emulsion Via Addition of a Chemical

In this example two collapsible emulsions were prepared and combined such that one contained the substrate and the other contained the enzyme and other reagents required for the reaction to proceed. The emulsion mixture was collapsed by addition of a chemical that destabilised the emulsion thus allowing the enzyme reaction to occur.

A TRITON X-114/mineral oil/octanol-based emulsion (referred to as the “starter” emulsion) was prepared as follows in a 2 ml microcentrifuge tube (Quantum Scientific) with vortexing on a VM1 vortex mixer (Ratek Instruments) at maximum setting. Forty microlitres of water was mixed with 10 microlitres of 10% (vol:vol or vol/vol) TRITON X-114 (Sigma-Aldrich). Eight hundred and seventy microlitres of mineral oil (Sigma) was added in small amounts with continuous vortexing of the sample. Care was taken to ensure that before each new addition of mineral oil the mixture had formed a homogenous emulsion. Thirty microlitres of n-octanol (Sigma-Aldrich) was added and the emulsion vortexed for 5 min.

The first emulsion, containing the DNA template, was prepared from 190 microlitres of starter emulsion and 2 micrograms of pGEM3Zf(+) (Amersham Pharmacia) contained in 10 microlitres of water. The emulsion was vortexed for 1 min. The second emulsion, containing the restriction enzyme and buffer, was prepared from: 760 microlitres of starter emulsion, 10 microlitres of water, 10 microlitres of 10× NEBuffer 1 (New England Biolabs, Inc. Mass., USA), and 10 microlitres of a 1 mg/ml BSA. The emulsion was vortexed for 1 min. Ten microlitres of Kpn I restriction enzyme (New England Biolabs) in 10 microlitres of water was added and the emulsion vortexed for 2 sec.

Three separate emulsion mixes were created from 40 microlitres of the DNA template emulsion and 160 microlitres of the restriction enzyme emulsion in 1.5 ml centrifuge tubes. The samples were not vortexed following addition of the second emulsion and prior to incubation to avoid mixing the two separate emulsions. Sample 1 was incubated at 37° C. for 1 h. The emulsion was observed to remain approximately 90% intact after incubation. Sample 2 was heated to 70° C. for 10 min followed by 37° C. for 1 h. This treatment collapsed the emulsion prior to incubation at 37° C. Sample 3 was collapsed by the addition of 10 microlitres of 100% glycerol (AR grade, Ajax Laboratory Chemicals, NSW, Australia) prior to the 1 h incubation at 37° C.

After incubation, the reactions were stopped and the DNA was recovered by the addition of 150 microlitres of buffered phenol (Sigma-Aldrich) and 100 microlitres of water. The samples were vortexed for 5 s before centrifugation at 14,000 g for 3 min. One hundred microlitres of the aqueous phases was transferred to a clean 1.5 ml centrifuge tubes containing 900 microlitres of n-butanol (Sigma-Aldrich). The tubes were vortexed for 10 s and the DNA pelleted by centrifugation at 14,000 g for 10 min. The DNA pellets were dried in a DNA110 Speed Vac (Savant, Waltham, Mass., USA) for 15 min before being re-dissolved in 20 microlitres of water.

The Kpn I enzyme cuts the circular pGE3Zf(+) plasmid at a single site converting it to a linear DNA fragment that displays a different mobility to the circular form under agarose gel electrophoresis allowing the progress of the reaction to be determined. Ten microlitres of each sample were run on a 0.8% (w/vol) agarose gel in 1× TAE buffer (40 mM Tris-acetate, 1 mM EDTA) before being stained with ethidium bromide and visualised under UV illumination.

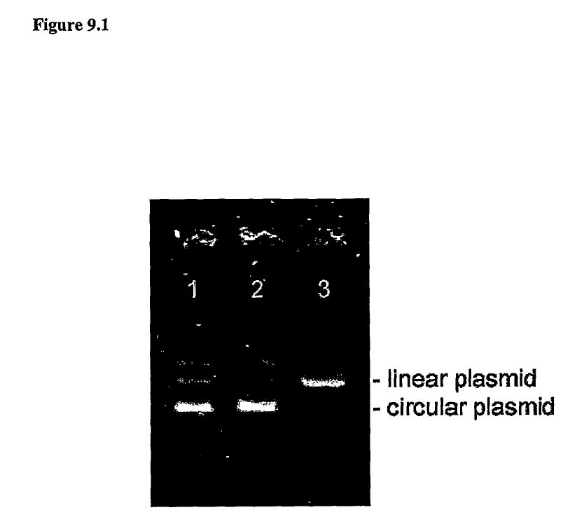

FIG. 9 shows that the restriction enzyme digestion reaction does not proceed efficiently in intact emulsions (lane 1), whereas the digest proceeds almost to completion (lane 3) when the emulsion is collapsed by the addition of glycerol. An additive such as glycerol is potentially useful as a means to collapse an emulsion in situations where thermal collapse of the emulsion leads to heat inactivation of the enzyme, as occurred with Sample 2 (lane 2).

This example demonstrates that when the substituents of two intact emulsions are combined together in a single tube they are essentially unable to mix together and the enzyme reaction is unable to proceed. These emulsions can be induced to interact via the addition of a chemical or by heat treatment. When the combined emulsions are collapsed to yield a substantially single aqueous phase, in which the essential components of the reaction can freely mix, the reaction can proceed. The collapse of the emulsion can therefore act as a switch to initiate the enzyme reaction. The use of a chemical or chemical mixture to collapse emulsions or emulsion mixtures allows the invention to be utilised in applications that cannot tolerate high temperatures e.g. thermolabile enzyme reactions.

### Example 10

### Combination of Collapsible Emulsion and Dilution Buffers to Reduce the Amount of Consumables in DNA Sequencing Reactions

This example shows that DNA sequencing in collapsible emulsions can be combined with dye terminator dilution buffers commonly used with DNA sequencing chemistry.

The DNA templates were prepared by adding 10 ng of HindIII linearized pUC18 plasmid DNA and 5 pmol of pGemEcoRV primer to a 0.2 ml PCR tube. The DNA was dried at 80° C. for 30 min.

The collapsible emulsion was prepared in a 2 ml microcentrifuge tube (Quantum Scientific) with vortexing on a VM1 vortex mixer (Ratek Instruments) at maximum setting. Forty microlitres of water was mixed with 10 microlitres of 10% (vol:vol) TRITON X-114 (Sigma-Aldrich). Eight hundred and fifty microlitres of mineral oil (Sigma-Aldrich) was added in small amounts (approximately 100 microlitres at a time) with continuous vortexing of the sample. Care was taken to ensure that before each new addition of mineral oil the mixture had formed a homogenous emulsion. Fifty microlitres of n-octanol (Sigma-Aldrich) was added and the emulsion vortexed for 5 mm.

Ninety-five microlitres of this emulsion was aliquoted into 1.5 ml microcentrifuge tubes (Product number 72.690; Sarstedt, Adelaide/Australia). To the emulsion 0.8 microlitres of BigDye Version 3 premix (Applied Biosystems) and 3.2 microlitres 2.5× dilution buffer (200 millimolar Tris-HCl (pH 9), 5 millimolar magnesium chloride and 1.5 molar sucrose) was added and mixed by repetitive pipetting (10-20 times) and vortexing for 1 s. Ten microlitres of the emulsion was added to the reaction tube containing the dried pUC18 plasmid DNA and pGemEcoRV primer.

The reaction was heated for 20 s at 96° C. before being cycled 99 times at 96° C. for 10 s, 45° C. for 30 s and 60° C. for 4 min. Excess labelled nucleotides were removed and the samples analysed as described in Example 6.

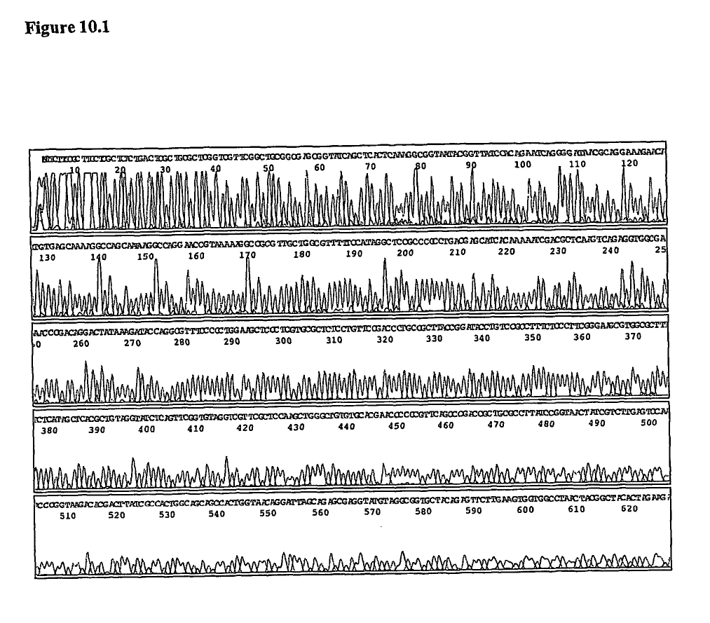

The resulting sequencing trace of the diluted sequencing reaction in a collapsible emulsion is shown in FIG. 10. High quality sequence data with readable data beyond position 600 was obtained (FIG. 10).

This example demonstrates that the dilution of sequencing chemistry in collapsible emulsions is possible. The reaction described in this example contains a final amount of 80 nanolitres (0.08 microlitres) of sequencing chemistry, which is at a 100-fold lower scale than that recommended by the manufacturer of the sequencing chemistry (Applied Biosystems 2001).

### Example 11

### Sequencing Reactions in a Collapsible Emulsion Using DNA Templates in Liquid

This example shows the performance of a sequencing reaction in a collapsible emulsion when the sequencing template is added in a liquid form. This application is helpful where the sequencing template cannot be dried down as described in Examples 6 or 10.

A DNA solution was prepared by adding 10 ng of HindIII linearized pUC18 plasmid DNA and 5 pmol of pGemEcoRV primer to a sucrose solution (final sucrose concentration 540 mM).

The collapsible emulsion was prepared in a 2 ml microcentrifuge tube (Quantum Scientific) with vortexing on a VM1 vortex mixer (Ratek Instruments) at maximum setting. Ten microlitres of 10% (vol:vol) TRITON X-114 (Sigma-Aldrich), 16 microlitres of Tris-HCl. (pH 9), four microlitres of 100 millimolar magnesium chloride and 20 microlitres of bovine serum albumin (0.1 mg/ml) were mixed. Eight hundred and sixty microlitres of mineral oil (Sigma-Aldrich) was added in small amounts (approximately 100 microlitres at a time) with continuous vortexing of the sample. Care was taken to ensure that before each new addition of mineral oil the mixture had formed a homogenous emulsion. Forty microlitres of n-octanol (Sigma-Aldrich) was added and the emulsion vortexed for 5 min.

Ninety-five microlitres of this emulsion was aliquoted into 1.5 ml microcentrifuge tubes (Product number 72.690; Sarstedt, Adelaide/Australia). To the emulsion 4 microlitres of BigDye Version 3 premix (Applied Biosystems) and 1 microlitre of sterile Milli-Q water was added and mixed by repetitive pipetting (10-20 times) and vortexing for 1 sec. Five microlitres of the emulsion was added into 0.2 ml PCR tubes. Then 1 microlitre of the DNA solution containing template and primer (see above) was pipetted into the emulsion.

The reaction was heated for 20 sec at 96° C. before being cycled 99 times at 96° C. for 10 s, 45° C. for 30 s and 60° C. for 4 min. Excess labelled nucleotides were removed and the samples analysed as described in Example 6.

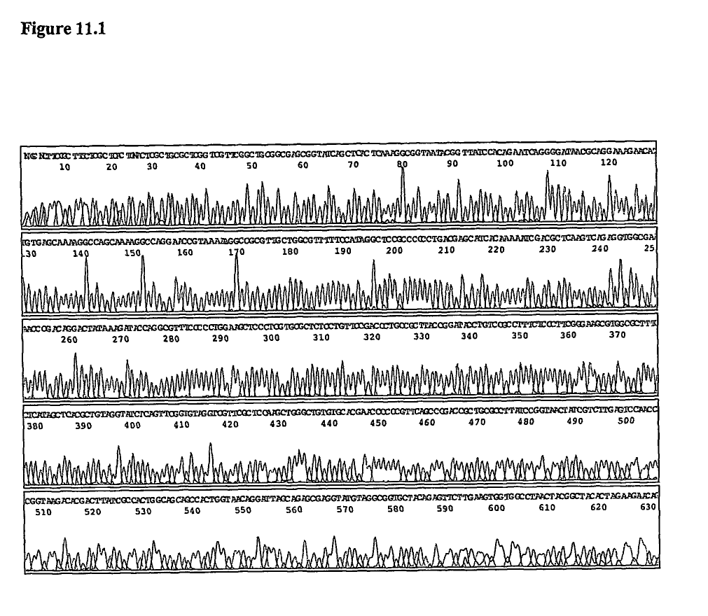

The resulting sequencing trace of the sequencing reaction in a collapsible emulsion with liquid template addition is shown in FIG. 11. High quality sequence data with readable data beyond position 600 was obtained (FIG. 11).

This example demonstrates that a liquid DNA template can be used for a DNA sequencing reaction in a small reaction phase when added to a collapsible emulsion containing sequencing chemistry. In comparison to Example 6 and 10 this example also shows that two different reactants (template and sequencing chemistry) in a collapsible emulsion reaction can be delivered and combined in different states (e.g. solid/dried or liquid).

### Example 12

### Effect of the Volume of the Inert Phase on the Sequencing Reaction in a Small Reaction Phase

Examples 6, 7 and 10 demonstrated DNA sequencing reactions in collapsible emulsion with the inert phase 9 times the volume of the reaction phase. This example describes an experiment to investigate what volume ratios between the reaction phase and the inert phase are suitable for sequencing reactions under the described conditions and with the sequencing chemistry used. For this example, mineral oil, which is the major component of the collapsible emulsion used in Example 6, 7 and 10, is used to overlay small, standard sequencing reactions. By varying the ratio of the volume of the inert phase to the reaction phase, potential inhibition of the sequencing reaction by the inert phase can be assessed.

The DNA samples were prepared by adding 10 ng of HindIII linearized pUC18 plasmid DNA and 5 pmol of pGemEcoRV primer to a 0.2 ml PCR tube. The DNA was dried at 80° C. for 30 min.

Four microlitres of BigDye Version 3 premix (Applied Biosystems) and 6 microlitres of sterile Milli-Q water were combined and 1 microlitre of this standard sequencing mix was added to the dried DNA sample (see above). Variable amounts (5, 10, 40 and 120 microlitres) of mineral oil were layered of the 1 microlitre reaction volume and spun briefly (10 sec, 1000× g) to ensure that the reaction phase was below the inert phase.

The reaction was heated for 20 s at 96° C. before being cycled 99 times at 96° C. for 10 s, 45° C. for 30 s and 60° C. for 4 min. Excess labelled nucleotides were removed and the samples analysed as described in Example 6.

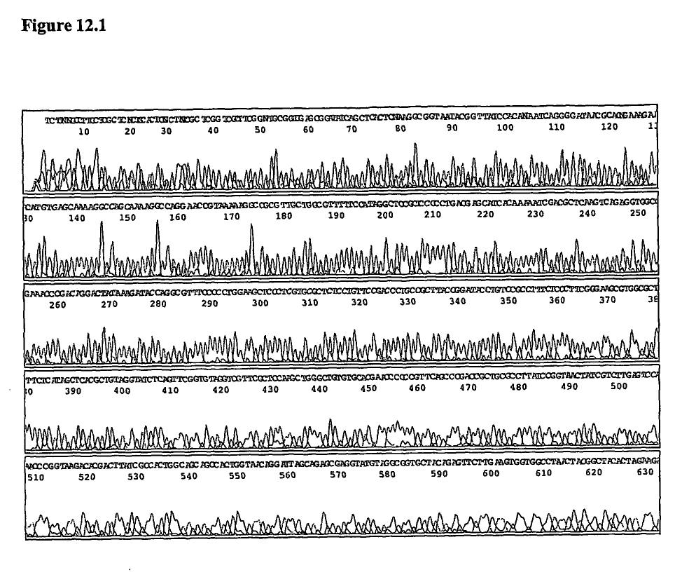

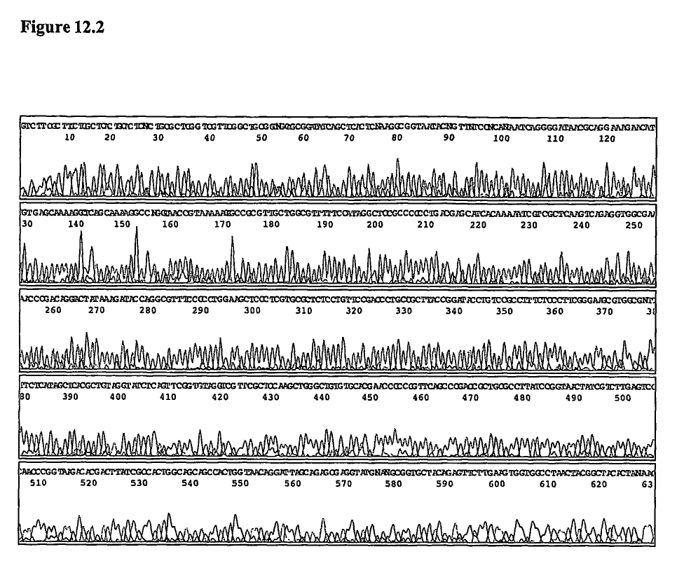

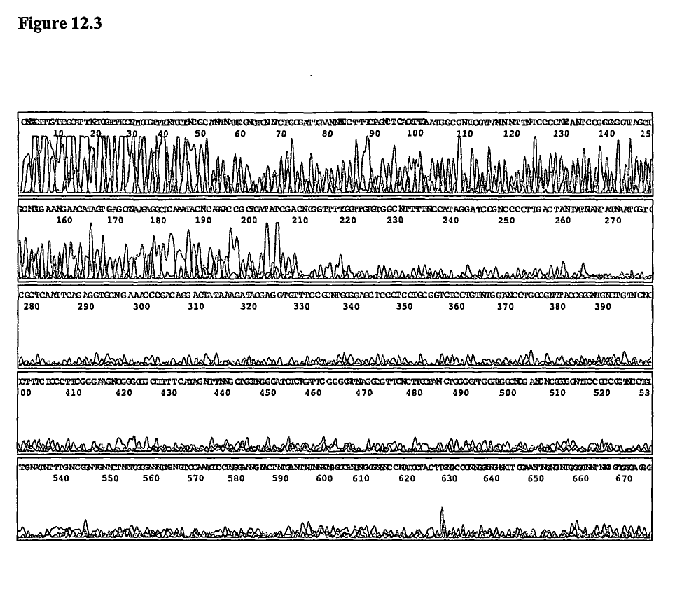

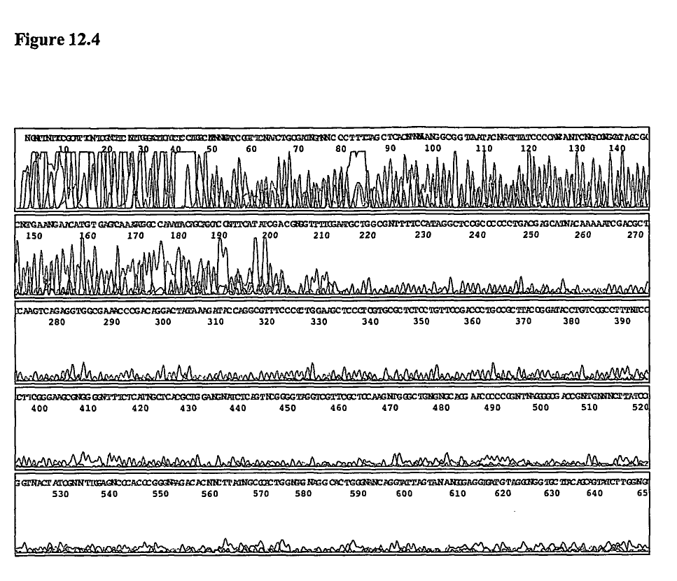

The resulting traces obtained from the DNA sequencing reaction using different volumes of mineral overlays are shown in FIG. 12.1 (5 microlitres overlay), FIG. 12.2 (10 microlitres overlay), FIG. 12.3 (40 microlitres overlay) and FIG. 12.4 (120 microlitres overlay). High quality sequence data with readable data beyond position 600 was obtained for the 5 microlitres and 10 microlitres mineral oil overlays (see FIG. 12.1 and 12.2, respectively). In contrast, the 40 microlitres and 120 microlitres overlays showed poor sequence quality with readable data only to approximately position 200 (see FIG. 12.3 and 12.4).

These data show that there may be a limit for suitable volume ratios between a given inert phase and a reaction phase for optimal reaction conditions. In this example volume ratios up to 10 for inert phase:reaction phase are suitable, while ratios of 40 and higher show inhibition of the sequencing reaction. Without wishing to be bound by theory, potential explanations for the inhibition observed include possible partitioning of either an inhibitory substance from the inert phase into the reaction phase or an activating substance from the reaction phase into the inert phase. With increased volume ratios the inhibitor or activator can reach concentration thresholds that have a negative impact on the reaction performed in the small, aqueous reaction phase.

Although the invention has been described with reference to specific examples, it will be appreciated by those skilled in the art that the invention may be embodied in many other forms.

In particular, the invention exemplified is an invention in which an aqueous phase dispersed in an inert phase in the form of an emulsion is collapsed to provide an aqueous reaction mixture. However, it will be abundantly clear to the skilled addressee that the phases may be inversed—that is to say, that the “bulking” agent could be an aqueous phase. In this situation, the reactants would be present initially in a discontinuous organic phase which discontinuous organic phase is present in a continuous aqueous phase. The aqueous phase would “collapse” to provide a substantially continuous organic phase in which the chemical reaction would take place.

### REFERENCES

Anarbaev, R. O., Elepov, I. B. & Lavrik, O. I. (1998). Klenow Fragment and DNA Polymerase Alpha-Primase from Serva Calf Thymus in Water-in-Oil Microemulsions. Biochimica et Biophysica Acta—Protein Structure & Molecular Enzymology 1384, 315-324.
Applied Biosystems (2001) Manual for ABI PRISM® BigDye™ Terminator v3.0 Ready Reaction Cycle Sequencing Kit (P/N 4390037 Rev. A)
Ausubel, F. M., Brent, R., Kingston, R. E., Moore, D. D., Seidman, J. G., Smith, J. A. & Struhl, K. (1998). Current Protocols in Molecular Biology. Edited by V. B. Chanda: John Wiley & Sons, Inc.
Azadan, R. J., Fogleman, J. C. & Danielson, P. B. (2002). Capillary electrophoresis sequencing: Maximum read length at minimal cost. Biotechniques 32, 24±.
Florence, A. T., Madsen, F. & Puisieux, F. (1975). Emulsion stabilization by non-ionic surfactants: the relevance of surfactant cloud point. 27, 385-94.
Gu, T. R. & Galera-Gomez, P. A. (1999). The effect of different alcohols and other polar organic additives on the cloud point of Triton X-100 in water. Colloids & Surfaces A—Physicochemical & Engineering Aspects 147, 365-370.
Hadd, A. G., Goard, M. P., Rank, D. R. & Jovanovich, S. B. (2000). Sub-microliter DNA sequencing for capillary array electrophoresis. Journal of Chromatography 894, 191-201.
He, Y., Pang, H. M. & Yeung, E. S. (2000). Integrated electroosmotically-driven on-line sample purification system for nanoliter DNA sequencing by capillary electrophoresis. Journal of Chromatography 894, 179-90.
Helenius, A., McCaslin, D. R., Fries, E. & Tanford, C. (1979). Properties of detergents. Methods in Enzymology 56, 734-49.
Leung, Y. F., Tam, P. O., Baum, L., Chan, W. M., Lam, D. S. & Pang, C. P. (2000). Cost savings using automated DNA sequencing. Biotechniques 29, 544.
Luisi, P. L., Giomini, M., Pileni, M. P. & Robinson, B. H. (1988). Reverse micelles as hosts for proteins and small molecules. Biochimica et Biophysica Acta 947, 209-46.
Meldrum, D. (2000). Automation for genomics, part two: sequencers, microarrays, and future trends. Genome Research 10, 1288-303.
Meldrum, D. R., Evensen, H. T., Pence, W. H., Moody, S. E., Cunningham, D. L. & Wiktor, P. J. (2000). ACAPELLA-1K, a capillary-based submicroliter automated fluid handling system for genome analysis. Genome Research 10, 95-104.
Nakane, J., Broemeling, D., Donaldson, R., Marziali, A., Willis, T. D., O'Keefe, M. & Davis, R. W. (2001). A method for parallel, automated, thermal cycling of submicroliter samples. Genome Research 11, 441-447.
Neugebauer, J. M. (1990). Detergents: an overview. Methods in Enzymology 182, 239-53.
Pang, H. M. & Yeung, E. S. (2000). Automated one-step DNA sequencing based on nanoliter reaction volumes and capillary electrophoresis. Nucleic Acids Research 28, E73.
Saiki, R. K., Gelfand, D. H., Stoffel, S., Scharf, S. J., Higuchi, R., Horn, G. T., Mullis, K. B. & Erlich, H. A. (1988). Primer-directed enzymatic amplification of DNA with a thermostable DNA polymerase. Science 239, 487-91.
Schott, H. & Han, S. K. (1977). Effect of symmetrical tetraalkylammonium salts on cloud point on nonionic surfactants. 66, 165-8.
Schott, H. & Royce, A. E. (1984). Effect of inorganic additives on solutions of nonionic surfactants VI: Further cloud point relations. 73, 793-9.
Schramm, L. L. (1993). The Language of Colloid and Interface Science; A Dictionary ofTerms, Washington, D.C.,: American Chemical Society; Professional Reference Books.
Shandrick, S., Ronai, Z. & Guttman, A. (2002). Rapid microwell polymerase chain reaction with subsequent ultrathin-layer gel electrophoresis of DNA. Electrophoresis 23, 591-595.
Shimura, K., Uchiyama, N. & Kasai, K. (2001). Prevention of evaporation of small-volume sample solutions for capillary electrophoresis using a mineral-oil overlay. Electrophoresis 22, 3471-7.
Soper, S. A., Williams, D. C., Xu, Y., Lassiter, S. J., Zhang, Y., Ford, S. M. & Bruch, R. C. (1998). Sanger DNA sequencing reactions performed in a solid-phase nanoreactor directly coupled to capillary gel electrophoresis. Analytical Chemistry 70, 4036-43.
Walde, P., Peng, Q., Fadnavis, N. W., Battistel, E. & Luisi, P. L. (1988). Structure and activity of trypsin in reverse micelles. European Journal of Biochemistry 173, 401-9.
Xue, G., Pang, H. M. & Yeung, E. S. (2001). On-line nanoliter cycle sequencing reaction with capillary zone electrophoresis purification for DNA sequencing. Journal of Chromatography 914, 245-56.

1. A method of performing a chemical reaction between reactants comprising:
(a) subjecting an emulsion comprising
(i) a discontinuous first phase in which at least one of the reactants is present; and
(ii) a substantially continuous second phase,
to a physical or chemical change such that a substantially continuous phase is formed from the discontinuous phase; and
(b) providing conditions in which the chemical reaction between the reactants takes place.

2. A method according to claim 1 wherein the discontinuous first phase is an aqueous phase.

3. A method according to claim 1 wherein the continuous second phase is an inert or an organic phase.

4. A method of performing a chemical reaction between reactants in an aqueous phase comprising:
(a) subjecting an emulsion comprising
(i) a discontinuous aqueous phase in which at least one of the reactants is present; and
(ii) a continuous inert phase,
to a physical or chemical change such that a substantially continuous aqueous phase is formed; and
(b) providing conditions in which the chemical reaction between the reactants takes place.

5. A method according to claim 1 or claim 4 wherein the chemical reaction is a reaction selected from the group consisting of: DNA sequencing, Polymerase Chain Reaction (PCR), Rolling Circle Amplification (RCA), Ligase chain Reaction (LCR), Rapid Amplification of cDNA Ends (RACE), reverse-transcriptase PCR (RT-PCR), DNA fingertyping, DNA genotyping, endonuclease-restriction digest, DNA ligation, DNA phosphorylation, DNA methylation, DNA labeling, ribonucleic acid (RNA) digestion, proteolytic digestion, and protein modification.

6. A method according to claim 1 or claim 4 wherein the chemical reaction is protein modification and wherein the protein modification is glycosylation or phosphorylation.

7. A method according to claim 1 or claim 4 wherein the chemical reaction is DNA sequencing or PCR.

8. A method according to claim 1 or claim 4 wherein the reactants are selected from the group consisting of: DNA, RNA, mRNA, proteins, enzymes, salts, radioactive isotopes and carbohydrates.

9. A method according to claim 1 or claim 4 wherein the reactants are selected from the group consisting of: gDNA, cDNA, mDNA, primer DNA, plasmid DNA or a PCR product.

10. A method according to claim 1 or claim 4 wherein the reactant is an enzyme and wherein the enzyme is a DNA polymerase, RNA polymerase, reverse transcriptase, restriction endonuclease, DNA methylase, polynucleotide kinase, nucleotide transferase, DNA ligase, RNA ligase, protease, or other DNA, RNA or protein modifying enzyme.

11. A method according to claim 2 or claim 4 wherein the aqueous phase is in a submicrolitre or microlitre volume.

12. A method according to claim 3 or claim 4 wherein the emulsion comprises a single inert phase and two or more different aqueous phases.

13. A method according to claim 1 or claim 4 wherein the emulsion is prepared by combining a first and second emulsion wherein
(a) the first emulsion comprises a first aqueous phase and a first inert phase wherein the first aqueous phase comprises a first reactant; and
(b) the second emulsion comprises a second aqueous phase and a second inert phase wherein the second aqueous phase comprises a second reactant.

14. A method according to claim 13 wherein the first and second inert phases are the same but the first and second aqueous phases are different.

15. A method according to claim 13 wherein the first insert phase and the second inert phase are different.

16. A method according to claim 3 or claim 4 wherein the inert phase is a non-polar water-immiscible compound or composition.

17. A method according to claim 3 or claim 4 wherein the inert phase is selected from the group consisting of: a hydrocarbon compound, a linear, branched or cyclic polysiloxane; a mineral or petroleum oil.

18. A method according to claim 3 or claim 4 wherein the inert phase is a hydrocarbon compound and wherein the hydrocarbon compound is selected from the group consisting of: pentane, hexane, heptane, octane, nonane, decane, dodecane, hexadecane, octadecane, eicosane, squalene and derivates thereof.

19. A method according to claim 3 or claim 4 wherein the inert phase is a hydrocarbon compound and wherein the hydrocarbon is selected from the group consisting of: 7-methyl-1,6-octadiene or 2,2,4-trimethylpentane, 1-dodecene, 1-hexadecane, cyclohexane and propylcyclohexane.

20. A method according to claim 3 or claim 4 wherein the inert phase is selected from the group consisting of: mineral oil, hexadecane, dodecane and n-hexane.

21. A method according to claim 1 or claim 4 wherein the emulsion comprises a surfactant.

22. A method according to claim 1 or claim 4 wherein the emulsion comprises a surfactant and wherein the surfactant is selected from the group of non-ionic surfactants consisting of: APO-10, APO-12, BRIJ-35, C8E6, C10E6, C10E8, C12E6, C12E8 (Atlas G2127), C12E9, C12E10 (Brij 36T), C16E12, C16E21, cyclohexyl-n-ethyl-beta-D-maltoside, cyclohexyl-n-hexyl-beta-D-maltoside, cyclohexyl-n-methyl-beta-D-maltoside, n-decanoylsucrose, n-decyl-beta-D-glucopyranoside, n-decyl-beta-D-maltopyranoside, n-decyl-beta-D-thiomaltoside, n-dodecanoylsucrose, n-dodecyl-beta-D-glucopyranoside, n-dodecyl-beta-D-maltoside, genapol C-100, genapol X-80, genapol X-100, HECAMEG, heptane-1,2,3-triol, n-heptyl-beta-D-glucopyranoside, n-heptyl-beta-D-thioglucopyranoside, LUBROL PX, MEGA-8 (ocatanoyl-N-methylglucamide), MEGA-9 (nonanoyl-N-methylglucamide), MEGA-10 (decanoyl-N-methylglucamide), n-nonyl-beta-D-glucopyranoside, Nonidet P-10 (NP-10), Nonidet P-40 (NP-40), n-nonyl-beta-D-glucopyranoside, Nonidet P-10 (NP-10), Nonidet P-40 (NP-40), n-octanoyl-beta-D-glucoslyamine (NOGA), n-octanoylsucrose, n-octyl-alpha-D-glucopyranoside, n-octyl-beta-D-glucopyranoside, n-octyl-beta-D-maltopyranoside, PLURONIC F-68, PLURONIC F-127, THESIT, TRITON X-100 (tert-C8-Ø-E9.6;like NP-40), TRITON X-100 hydrogenated, TRITON X-114 (tert-C8-Ø-E7-8), TWEEN 20 (C12-sorbitan-E20;Polysorbate 20), TWEEN 40 (C16-sorbitan-E20), TWEEN 60 (C18-sorbitan-E20), TWEEN 80 (C18:1-sorbitan-E20), n-undecyl-beta-D-maltoside, cetearyl alcohol, hydrogenated tallow alcohol, lanolin alcohols, palmamide, peanutamide MIPA, PEG-50 tallow amide, cocamidopropylamine oxide, lauramine oxide, PEG-8 dilaurate, PEG-8 laurate, PEG-4 caster oil, PEG-120 glyceryl myristate, glyceryl palmitate lactace, polyglyceryl-6 distearate, polyglyceryl-4 oleyl ether, methyl gluceth-20 sesquisterase, sucrose disterate, polysorbate-60, sorbitan sequiisostearate, trideceth-3 phosphate, trioleth-8 phosphate, ceteareth-10, nonoxynol-9, PEG-20 lanolin, PPG-12-PEG-65 lanolin oil, dimethicone copolyol, meroxapol 314, poloxamer 122, PPG-5-cetech-20 and lauryl glucose.

23. A method according to claim 1 or claim 4 wherein the emulsion comprises a surfactant and wherein the surfactant is selected from the group of ionic surfactants consisting of: caprylic acid (n-octanoate), cetylpyridinium chloride, CTAB (Cetyltri-methylammonium bromide), cholic acid, decanesulfonic acid, deoxycholic acid, dodecyltrimethyl-ammonium bromide, glycocholic acid, glycodeoxycholic acid, lauroylsarcosine (sarkosyl), lithium n-dodecyl sulfate, lysophosphatidyl-choline, sodium n-dodecyl sulfate (SDS, lauryl sulfate), taurochenodeoxy-cholic acid, taurocholic acid, taurodehydrocholic acid, taurodeoxycholic acid, taurolithocholic acid, tauroursodeoxycholic acid, tetradecyltrimethyl-ammonium bromide (TDTAB), TOPPS, di-TEA-palmitoyl aspartate, sodium hydrogenated tallow glutamate, palmitoyl hydrolysed milk protein, sodium cocoyl hydrolysed soy protein, TEA-abietoyl hydrolysed collagen, TEA-cocoyl hydrolysed collagen, myristoyl sarcosine, TEA-lauroyl sarcosinate, sodium lauroyl taurate, sodium methyl cocoyl taurate, lauric acid, aluminum stearate, cottonseed acid, zinc undecylenate, calcium stearoyl lactylate, laureth-6 citrate, nonoxynol-8 carboxylic acid, sodium trideceth-13 carboxylate, DEA-oleth-10 phosphate, dilaureth-4 phosphate, lecithin, sodium cocoyl isethionate, sodium dodecylbenzene solfonate, sodium cocomonoglyceride sulfonate, sodium C12-14 olefin sulfonate, sodium C12-15 pareth-15 sulfonate, sodium lauryl solfoacetate, dioctyl sodium sulfosuccinate, disodium oleamido MEA-sulfosuccinate, ammonium laureth sulfate, sodium C12-13, pareth sulfate, MEA-lauryl sulfate, cocamidopropyl dimethylamine lactate, dimethyl lauramine, soyamine, stearyl hydroxyethyl imidazoline, PEG-cocopolyamine, PEG-15 tallow amine, benzalkonium chloride, quaternium-63, oleyl betaine, sodium lauramidopropyl hydroxyphostaine, cetylpyridinium chloride, isostearyl ethylimidonium ethosulfate, cocamidopropyl ethyldimonium ethosulfate, hydroxyethyl cetyldimonium chloride, quaternium-1 8 and cocodimonium hydroxypropyl hydrolysed hair keratin.

24. A method according to claim 1 or claim 4 wherein the emulsion comprises a surfactant and wherein the surfactant is selected from the goup of zwitterionic surfactants consisting of: BigCHAP, CHAPS, CHAPSO, DDMAU, EMPIGEN BB (N-dodecyl-N,N-dimethylglycine), lauryldimethylamine oxide (LADAO, LDAO, Empigen OB), SWITTERGENT 3-08, SWITTERGENT 3-10, ZWITTERGENT 3-12 (3 -dodecyl-dimethylammonio-propane-1-sulfonate), ZWITTERGENT 3-14, ZWITTERGENT 3-16, disodium cocoamphocarboxymethylhydroxy-propylsulfate, disodium cocoamphodipropionate, sodium cocoamphoacetate, sodium lauroampho PG-acetate phosphate, sodium tallow amphopropionate, sodium undecylenoamphopropionate, aminopropyl laurylglutamide, dihydroxyethyl soya glycinate and lauraminopropionic acid.

25. A method according to claim 1 or claim 4 wherein the emulsion comprises TRITON X-100 or TRITON-X114.

26. A method according to claim 1 or claim 4 wherein the physical or chemical change is a change in temperature, pressure or exposure to a chemical compound.

27. A method according to claim 1 or claim 4 wherein the physical change is a change in temperature.

28. A method according to claim 1 or claim 4 wherein the chemical change is the addition of glycerol.

29. A method according to claim 4 wherein when the chemical reaction is a DNA sequencing or PCR reaction, the inert phase comprises mineral oil and the surfactant is TRITON X-100 or TRITON-X114.

30. A method according to claim 1 or claim 4 wherein the ratio of the aqueous to inert phase is in the range of 1:4 to 1:19.

31. A method according to claim 1 or claim 4 wherein the inert phase is removed from the substantially continuous aqueous phase after the chemical reaction has taken place.

32. A method according to claim 1 or claim 4 wherein the inert phase is removed from the substantially continuous aqueous phase by suction or evaporation.

33. A method according to claim 3 or claim 4 wherein the aqueous phase and the inert phase are submitted to the reaction conditions together.

34. A method of performing a chemical reaction between at least two reactants in an aqueous solution comprising:
(a) combining a first emulsion in which an aqueous solution comprising a first reactant is emulsified in a first inert phase, with a second emulsion in which an aqueous solution comprising a second reactant is emulsified in a second inert phase;
(b) subjecting the mixture to a physical or chemical change such that the emulsions collapse and the emulsified aqueous solution coalesces into a substantially single or substantially continuous aqueous phase;
(c) subjecting the aqueous phase to conditions in which the chemical reaction between the reactants take place.

35. A method of performing a chemical reaction between reactants in an organic phase comprising:
(a) subjecting an emulsion comprising
(i) a discontinuous organic phase in which at least one of the reactants is present; and
(ii) a continuous aqueous phase,
to a physical or chemical change such that a substantially continuous organic phase is formed; and
(b) providing conditions in which the chemical reaction between the reactants takes place.

36. A method of performing a chemical reaction between at least two reactants in an organic solution comprising:
(a) combining a first emulsion in which an organic solution comprising a first reactant is emulsified in a first aqueous phase, with a second emulsion in which an organic solution comprising a second reactant is emulsified in a second aqueous phase;
(b) subjecting the mixture to a physical or chemical change such that the emulsions collapse and the emulsified organic solution coalesces into a substantially single or substantially continuous organic phase.
(c) subjecting the organic phase to conditions in which the chemical reaction between the reactants takes place.


SEQUENCE LISTING (11 sequences)

SEQ ID NO: 1 - DNA, 4245 bp, Artificial sequence
  Plasmid pCR-Blunt II-TOPO
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccctcatatgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttcgcgtatggtcttcaatgctttgcgagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataaggatcctaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - DNA, 3197 bp, Artificial sequence
  Plasmid pGEM-3Zf (+)
gggcgaattcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 3 - DNA, 7249 bp, Artificial sequence
  Bacteriophage M13mp18
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcgctggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatacggtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 4 - DNA, 1016 bp, Artificial sequence
  PCR product
agcggataacaatttcacacaggaaacagctatgaccatgattacgccaagctatttaggtgacactatagaatactcaagctatgcatcaagcttggtaccgagctcggatccactagtaacggccgccagtgtgctggaattcgccctcatatgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttcgcgtatggtcttcaatgctttgcgagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataaggatcctaagggcgaattctgcagatatccatcacactggcggccgctcgagcatgcatctagagggcccaattcgccctatagtgagtcgtattacaattcactggccgtcgttttacaacgtcgtgactgggaaaaccctggcg

SEQ ID NO: 5 - DNA, 2686 bp, Artificial sequence
  Plasmid pUC18
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 17 bp, Artificial sequence
  Synthetic primer
actggccgtcgttttac

SEQ ID NO: 7 - DNA, 16 bp, Artificial sequence
  Synthetic primer
aacagctatgaccatg

SEQ ID NO: 8 - DNA, 24 bp, Artificial sequence
  Synthetic primer
cgccagggttttcccagtcacgac

SEQ ID NO: 9 - DNA, 24 bp, Artificial sequence
  Synthetic primer
agcggataacaatttcacacagga

SEQ ID NO: 10 - DNA, 16 bp, Artificial sequence
  Synthetic primer
gtaaaacgacggccag

SEQ ID NO: 11 - DNA, 19 bp, Artificial sequence
  Synthetic primer
atcgcggtttgcgtattgg